(19) United States
(12) Patent Application Publication
(10) Pub. No.: US 2024/0018230 A1
(43) Pub. Date: Jan. 18, 2024

(54) METHODS FOR TREATING OR MODULATING AN INFLAMMATORY RESPONSE

(71) Applicant: St. Jude Children's Research Hospital, Inc., Memphis, TN (US)

(72) Inventors: Thirumala-Devi KANNEGANTI, Memphis, TN (US); Rajendra KARKI, Memphis, TN (US)

(21) Appl. No.: 18/245,257

(22) PCT Filed: Sep. 21, 2021
(86) PCT No.: PCT/US2021/051206

§ 371 Date: Mar. 14, 2023

Related U.S. Application Data
(60) Provisional application No. 63/080,819, filed on Sep. 21, 2020.
(60) Provisional application No. 63/106,012, filed on Oct. 27, 2020.

(51) Int. Cl. C07K 16/24; A61P 37/06

(52) U.S. Cl. CPC C07K 16/241; C07K 16/249; A61P 37/06; A61K 2039/507

(57) ABSTRACT

Methods for treating or mitigating COVID-19, sepsis or hemophagocytic lymphohistiocystosis or cytokine storm-associated syndromes; and treating or preventing inflammatory cell death associated with an inflammatory condition or infection such as SARS-CoV-2, or inducing inflammatory cell death in the context of cancer are provided, which includes the modulation of TNF and/or IFN-γ activity.

### INTRODUCTION

This patent application claims the benefit of priority from U.S. Provisional Ser. No. 63/080,819, filed Sep. 21, 2020, and 63/106,012, filed Oct. 27, 2020, the contents of which are incorporated herein by reference in their entireties.

This invention was made with government support under Grant Numbers AI101935, AI124346, AR056296, A1160179, CA163507 and CA253095 awarded by the National Institutes of Health. The government has certain rights in the invention.

### BACKGROUND

The activation of the immune system and production of inflammatory cytokines are essential for normal anti-pathogen immune responses. Cytokines are produced by several immune cells including the innate immune cells, macrophages, dendritic cells, and natural killer cells, and the adaptive immune cells, T and B lymphocytes, during infection. However, aberrant immune system activation results in a sudden acute increase in circulating levels of pro-inflammatory cytokines and interferons, leading to an event known as a “cytokine storm,” defined as a life-threatening condition caused by excessive production of cytokines mediated by inflammatory cell death, PANoptosis, characterized by a clinical presentation of systemic inflammation, hyperferritinemia, hemodynamic instability, and multi-organ failure.

The newly emerging coronavirus disease 2019 (COVID-19), caused by severe respiratory syndrome coronavirus 2 (SARS-CoV-2), has been a challenge to the global healthcare system. SARS-CoV-2 infection results in a broad spectrum of clinical manifestations, including both asymptomatic cases and rapid fatalities. While SARS-CoV-2 is a respiratory pathogen, patients present with systemic symptoms of varying severity. This, is associated with an aggressive inflammatory response and the release of a large amount of pro-inflammatory cytokines. Patients with severe COVID-19, both in and out of the intensive care unit, have elevated levels of circulating IL-1β, IL-7, IL-8, IL-9, IL-10, FGF, G-CSF, GM-CSF, IFN-γ, IP-10, MCP-1, MIP-1α, MIP1-β, PDGF, TNF-α, and VEGF. Several studies analyzing cytokine profiles from patients with COVID-19 have suggested a direct correlation between the cytokine storm and lung injury, multiple organ failure, and unfavorable prognosis (Jamilloux, et al. (2020) Autoimmun. Rev. 19(7):102567; Zhang, et al. (2020) Life Sci. 250:117583; Harrison (2020) Nature Biotechnol. 38:905-916). However, a mechanistic understanding of how targeting cytokines could be beneficial or which cytokines are involved in disease pathogenesis is completely lacking. Additionally, there was initially some controversy as to whether the phenomenon observed during COVID-19 is in fact a cytokine storm, as emerging evidence showed that the levels of proinflammatory cytokines in these patients are much lower than in patients with other diseases associated with cytokine storm, such as sepsis (Leisman et al. (2020) Lancet Respir. Med.; Kox et al. (2020) JAMA 324(15):1565-1567). However, another similar study found increased expression of TNF and IL-1 in PBMCs of patients with COVID-19 compared to patients with influenza (Lee et al. (2020) Sci. Immunol. 5(49):eabd1554). Acute respiratory distress syndrome (ARDS) is one of the serious consequences of the increased cytokine levels in COVID-19. Understanding the underlying mechanisms driving the development of ARDS and multiple organ failure during COVID-19 and identifying whether cytokine storm is responsible for the pathology is critical to identify why certain sub-populations are more likely to die of COVID-19 than others. Virus is found in the lungs and airways early in infection but not as the disease progresses, which suggests that elevated cytokine production is the driver of severe and critical conditions. Despite these findings, little is known about what cellular and molecular mechanisms contribute to lung damage and multiple organ failure in response to the increased cytokine production during SARS-CoV-2 infection.

Several other infections and inflammatory diseases are also associated with cytokine storm. These include but are not limited to bacterial sepsis, hemophagocytic lymphohistiocytosis (HLH), colitis, and inflammatory bowel disease (IBD). Understanding how cytokine storm drives inflammation in these disorders is also essential.

Conversely, activating inflammatory cell death in the context of cancer will be beneficial to eliminate tumor cells.

### SUMMARY OF THE INVENTION

This invention provides a method for treating or mitigating COVID-19 by administering to a subject in need of treatment an effective amount of a Tumor Necrosis Factor (TNF) inhibitor (e.g., infliximab, adalimumab, certolizumab, golimumab, etanercept, thalidomide, lenalidomide, pomalidomide, pentoxifylline, bupropion, or delmitide) and an Interferon gamma (IFN-γ) inhibitor (e.g., emapalumab, fontolizumab, AMG 811, vidofludimus, or delmitide). A kit composed of a TNF inhibitor and an IFN-γ inhibitor for use in treating or mitigating COVID-19 is also provided.

This invention also provides methods for treating or preventing inflammatory cell death associated with an infection or inflammatory condition by administering to a subject with an infection an effective amount of a TNF inhibitor (e.g., infliximab, adalimumab, certolizumab, golimumab, etanercept, thalidomide, lenalidomide, pomalidomide, pentoxifylline, bupropion, or delmitide), an IFN-γ inhibitor (e.g., emapalumab, fontolizumab, AMG 811, vidofludimus, or delmitide), or a combination thereof. In some aspects, the infection is a bacterial infection, a fungal infection, a parasitic infection, or a viral infection, e.g., an influenza infection or a coronavirus infection such as SARS-CoV-2. In other aspects, the inflammatory condition is sepsis or hemophagocytic lymphohistiocytosis. A kit composed of a TNF inhibitor and an IFN-γ inhibitor for use in treating or preventing inflammatory cell death associated with an infection is also provided.

### BRIEF DESCRIPTION OF THE DRAWINGS

FIG. 1 shows blocking TNF-α and IFN-γ provides protection in an animal model of SARS-CoV-2 infection. Shown is the survival of 7- to 8-week-old K18-hACE2 transgenic mice injected with isotype control (n=12) or neutralizing antibodies (Ab) against TNF-α and IFN-γ (n=13) on day 1, 3, and 4 after infection with SARS-CoV-2 (2×10⁴ pfu/mouse).

FIG. 2 shows blocking TNF-α and IFN-γ provides protection in an animal model of hemophagocytic lymphohistiocytosis (HLH). Shown is the survival of 7 to 8-week-old wild-type mice injected intraperitoneally with poly I:C (10 mg/kg body weight) followed 24 hours later by intraperitoneal injection of PBS (n=15), isotype control (n=15), neutralizing antibody against TNF-α (n=15), neutralizing antibody against IFN-γ (n=15), or neutralizing antibodies against both TNF-α and IFN-γ (n=15). Mice were then challenged with LPS (5 mg/kg body weight) 1 hour after the treatments.

FIG. 3 shows blocking TNF-α and IFN-γ provides protection in an animal model of sepsis. Shown is the survival of 7 to 8-week-old wild-type mice injected with PBS (n=17), isotype control (n=10), neutralizing antibody against TNF-α (n=17), neutralizing antibody against IFN-γ (n=17), or neutralizing antibodies against both TNF-α and IFN-γ (n=18) 30 minutes and 6 hours after intraperitoneal injection of a lethal dose of LPS (20 mg/kg body weight).

FIG. 4 shows time-course analysis of cell death of HT-29 cancer cells treated with TNF-α alone, IFN-γ alone, or TNF-α plus IFN-γ, assessed over the course of 48 hours post-stimulation. Data are representative of three independent experiments. Data are presented as the mean±SEM. ****P<0.0001. Analyses were performed using the two-way ANOVA.

FIG. 5 shows time-course analysis of cell death of SW-620 cancer cells treated with TNF-α alone, IFN-γ alone, or TNF-α plus IFN-γ, assessed over the course of 48 hours post-stimulation. Data are representative of three independent experiments. Data are presented as the mean±SEM. ****P<0.0001. Analyses were performed using the two-way ANOVA.

FIG. 6 shows time-course analysis of cell death of HCC2998 cancer cells treated with TNF-α alone, IFN-γ alone, or TNF-α plus IFN-γ, assessed over the course of 48 hours post-stimulation. Data are representative of three independent experiments. Data are presented as the mean±SEM. ****P<0.0001. Analyses were performed using the two-way ANOVA.

FIG. 7 shows time-course analysis of cell death of COLO-205 cancer cells treated with TNF-α alone, IFN-γ alone, or TNF-α plus IFN-γ, assessed over the course of 48 hours post-stimulation. Data are representative of three independent experiments. Data are presented as the mean±SEM. ****P<0.0001. Analyses were performed using the two-way ANOVA.

FIG. 8 shows time-course analysis of tumor volume of transplanted COLO-205 cell tumors in NSG mice treated with TNF-α alone, IFN-γ alone, or TNF-α plus IFN-γ. Data are presented as the mean±SEM. ****P<0.0001. Analyses were performed using the one-way ANOVA.

### DETAILED DESCRIPTION OF THE INVENTION

It has now been found that TNF and IFN-γ are the primary cytokines of the cytokine storm, which contribute to damage of vital organs in COVID-19. In particular, TNF and IFN-γ drive the inflammatory cell death response in COVID-19 by activating pyroptosis, apoptosis, and necroptosis, i.e., PANoptosis. Notably, neutralization of both TNF-α and IFN-γ in multiple disease models associated with cytokine shock was found to provide substantial protection against not only SARS-CoV-2 infection, but also sepsis, hemophagocytic lymphohistiocytosis and cytokine shock models. In contrast, the coadministration of TNF-α and IFN-γ induce cell death in human cancer cell lines and suppress the growth of transplanted xenograft tumors in an animal model. Accordingly, the present invention provides for modulating cell death by antagonizing or agonizing TNF and/or IFN-γ. In some aspects, the invention provides methods of treating or mitigating COVID-19, or more generally inhibiting inflammatory cell death or PANoptosis associated with an infection or inflammatory condition by administering to a subject an effective amount of a TNF inhibitor and/or an IFN-γ inhibitor or a combination of cytokine, signaling and effector molecule inhibitors, which limits further tissue damage including, but not limited to lung damage and multiple organ failure. In other aspects, the invention provides methods for treating cancer by inducing cell death via the administration of a TNF agonist and/or an IFN-γ agonist.

### Methods and Kits for Inhibiting Inflammatory Cell Death.

In accordance with the methods pertaining to inhibiting inflammatory cell death, a subject having, suspected of having, or at risk of having an infection, e.g., a bacterial or viral infection such as influenza or SARS-CoV-2, or inflammatory condition, e.g., sepsis, HLH, colitis or Crohn's disease, is administered an effective amount of a TNF inhibitor and/or an IFN-γ inhibitor to effect treatment or a reduction or mitigation in the severity of the infection or inflammation. In some aspects, therapy is initiated after the appearance of clinical signs of an infection or inflammatory condition. For example, in the case of SARS-CoV-2 therapy is initiated after the appearance of a fever exceeding 38° C., cough, fatigue, shortness of breath, muscle or body aches, headache, loss of taste or smell, and/or sore throat. In some aspects, therapy is administered prophylactically to the individual suspected of having an infection, e.g., a subject who is asymptomatic and not infected or yet infected, but has come into contact with an individual who has been diagnosed with an infection such as SARS-CoV-2; a subject who is asymptomatic but is diagnosed with an infection such as SARS-CoV-2; a subject who is expected to come into contact with individuals who have been diagnosed (e.g., health workers working in a facility where an individual who has been diagnosed with an infection such as $ARS-CoV-2); or a subject who is traveling to a place where a relatively high onset is known.

Whether administered before or after the development of clinical signs of infection or inflammation, administration of a composition of the invention can reduce the risk that the subject will develop severe symptoms (e.g., inflammatory cell death) associated with an infection or inflammatory condition (e.g., SARS-CoV-2, sepsis or HLH). In this respect, an effective amount of a TNF inhibitor and/or an IFN-γ inhibitor is an amount that alone or in a combination therapy reduces the risk or propensity for an individual to develop severe symptoms associated with an inflammatory condition or infection such as SARS-CoV-2 (e.g., hyperimmune response, clinical morbidity or mortality). For example, an effective amount is an amount that reduces the risk of developing severe symptoms by at least about 10%, at least about 20%, at least about 25%, at least about 30%, at least about 35%, at least about 40%, at least about 50%, at least about 60%, at least about 70%, at least about 80%, at least about 90% compared to the risk of developing severe symptoms associated with an inflammatory condition or infection such as SARS-CoV-2, sepsis or HLH in the absence of TNF inhibitor and/or IFN-γ inhibitor therapy. Whether severity decreases can be determined by measuring, e.g., cytokine or effector level or activity, or any symptom associated with the disease or conditions such as a coronavirus infection including, for example, respiratory symptoms (e.g., cough, easy or difficult breathing), and the like. Treatment or a reduction in severity can shorten the time the subject is sick, decrease reliance on a ventilator, decrease the time the subject is hospitalized, attenuate the development of ARDS, attenuate the development of multi-organ failure, etc.

The term “TNF inhibitor” refers to any compound or agent which reduces expression or production of TNF, directly binds to and reduces the activity of or degrades TNF, or blocks binding of TNF to its receptor, TNFR1 and TNFR2. Thus, a “TNF inhibitor” may broadly be regarded as any compound or agent which antagonizes or inhibits or reduces or prevents TNF activity. Suitable inhibitors may be oligonucleotide inhibitors of TNF expression, anti-TNF antibodies or small molecule inhibitors of TNF activity. A number of agents are known to inhibit TNF in humans including, e.g., anti-TNF antibodies such as infliximab, adalimumab, certolizumab, and golimumab (Lim, et al. (2018) Int. J. Mol. Sci. 19(3):768); protein-based therapeutics such as etanercept (WO 1991/003553), r-hTBP-1 (Onercept; Terlizzese, et al. (1996) J. Interferon Cytokine Res. 16:1047-53) and delmitide (Schall, et al. (2012) J. Autoimmun. 39:143-153); and small molecules such as thalidomide, lenalidomide and pomalidomide (Liu, et al. (2017) Exp. Thex. Med. 14(6):5251-7), pentoxifylline (or oxpentifylline; Dubost, et al. (1997) Rev. Rhum. Engl. Ed. 64(12):789-93), or bupropion (Hajhashemi & Khanjani (2014) Res. Pharm. Sci. 9(4):251-7). Any of the above agents, or combination of the above may be used as TNF inhibitors according to the present invention. Additional TNF inhibitors are described in U.S. Pat. Nos. 6,090,382, 6,258,562, 6,509,015, and 8,129,537.

Preferred TNF inhibitors include those which bind directly to TNF and inhibit the activity of the same. Preferably, the TNF inhibitor is an anti-TNF antibody or protein-based therapeutic. More preferably, the TNF inhibitor is infliximab, adalimumab, certolizumab, golimumab or etanercept.

The term “IFN-γ inhibitor” refers to any compound or agent which reduces expression or production of IFN-γ, directly binds to and reduces the activity of or degrades IFN-γ, or blocks binding of IFN-γ to its receptor. Thus, a “IFN-γ inhibitor” may broadly be regarded as any compound or agent which antagonizes or inhibits or reduces or prevents IFN-γ activity. Suitable inhibitors may be oligonucleotide inhibitors of IFN-γ expression, anti-IFN-γ antibodies or small molecule inhibitors of IFN-γ activity. A number of agents are known to inhibit IFN-γ in humans including, e.g., anti-IFN-γ antibodies such as emapalumab (Hommes, et al. (2006) Gut 55(8):1131-1137), fontolizumab (Reinisch, et al. (2010) Inflamm. Bowel Dis. 16(2):233-242), and AMG 811 (WO 2013/078378); protein-based therapeutics such as delmitide (Travis, et al. (2005) Inflamm. Bowel Dis. 11(8):713-179); and small molecules such vidofludimus (Muehler, et al. (2019) Drugs RD 19(4):351-66). Any of the above agents, or combination of the above may be used as IFN-γ inhibitors according to the present invention. Additional IFN-γ inhibitors are described in U.S. Pat. Nos. 8,906,371 and 6,830,752.

Preferred IFN-γ inhibitors include those which bind directly to IFN-γ and inhibit the activity of the same. Preferably, the IFN-γ inhibitor is an anti-IFN-γ antibody. More preferably, the IFN-γ inhibitor is emapalumab, fontolizumab or AMG 811.

The present invention also provides kits for carrying out the methods of treating or mitigating COVID-19 and treating or preventing inflammatory cell death associated with an infection or inflammatory condition. The kits of the invention include at least one TNF inhibitor in an effective amount and at least one IFN-γ inhibitor in an effective amount. The kits may provide a single dose or multiple doses of TNF inhibitor(s) and IFN-γ inhibitor(s), wherein said inhibitors may be provided individually or in a co-formulation. The kit may take the form of a blister package; a lidded blister, a blister card or packet; a clamshell; an intravenous (IV) package, IV packette or IV container, a tray or a shrink wrap comprising the inhibitors and instructions for use of the composition for treating or mitigating COVID-19 or treating or preventing inflammatory cell death associated with an infection such as SARS-CoV-2 or an inflammatory condition.

### Methods and Kits for Inducing Inflammatory Cell Death.

In accordance with the methods pertaining to inducing or activating inflammatory cell death and treating cancer, a subject having, suspected of having, or at risk of having cancer or other disease or condition that would benefit from inflammatory cell death (e.g., a proliferative disease or condition), is administered an effective amount of a TNF agonist and/or an IFN-γ agonist to effect treatment or reduction or mitigation of the cancer or other disease or condition. In some aspects, therapy is initiated after the appearance of clinical signs and/or symptoms of cancer. In other aspects, the treatment or reduction or mitigation of the cancer or other disease or condition is affected by the administration of a TNF agonist and an IFN-γ agonist in the absence of any other therapeutic agent. In a further aspect, the treatment or reduction or mitigation of the cancer or other disease or condition is affected by the intratumoral administration of a TNF agonist and an IFN-γ agonist in the absence of any other therapeutic agent. As demonstrated herein, coadministration of a TNF agonist (e. g., TNF-α protein) and a IFN-γ agonist (e.g., IFN-γ protein) provides a synergistic increase (i.e., more than additive increase) in cancer cell death and synergistic decrease in tumor volume.

As used herein, the term “symptom” is defined as an indication of disease, illness, injury, or that something is not right in the body. Symptoms are felt or noticed by the individual experiencing the symptom, but may not easily be noticed by others, e.g., non-health-care professionals. The term “sign” is also defined as an indication that something is not right in the body. But signs are defined as things that can be seen by a doctor, nurse, or other health care professional.

Cancer is a group of diseases that may cause a variety of signs or symptoms. The signs and symptoms will depend on where the cancer is, the size of the cancer, and how much it affects the nearby organs or structures. If a cancer spreads (metastasizes), then symptoms may appear in different parts of the body. As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms. A cancer may also cause symptoms such as fever, fatigue, or weight loss. This may be because cancer cells use up much of the body's energy supply or release substances that change the body's metabolism. Or the cancer may cause the immune system to react in ways that produce these symptoms. For example, fever may be an early sign of cancer, such as with leukemia or lymphoma. Fatigue may happen early in cancers such as with leukemia, or if the cancer is causing an ongoing loss of blood, as in some colon or stomach cancers. Sometimes, cancer cells release substances into the bloodstream that cause symptoms not usually thought to result from cancers. For example, some lung cancers make hormone-like substances that affect blood calcium levels, affecting nerves and muscles and causing weakness and dizziness. Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung. Alternatively, or in addition, cancer subtypes present specific signs or symptoms. Changes in bowel habits or bladder function could indicate cancer. Long-term constipation, diarrhea, or a change in the size of the stool may be a sign of colon cancer.

Whether administered before or after the development of clinical signs and/or symptoms of cancer, administration of a TNF agonist and/or an IFN-γ agonist can reduce the signs or symptoms of cancer. In this respect, an effective amount of a TNF agonist and/or an IFN-γ agonist is an amount that alone or in a combination therapy can, e.g., reduce or inhibit tumor growth. For example, an effective amount is an amount that reduces tumor growth by at least about 10%, at least about 20%, at least about 25%, at least about 30%, at least about 35%, at least about 40%, at least about 50%, at least about 60%, at least about 70%, at least about 80%, at least about 90% compared to tumor growth in the absence of TNF agonist and/or an IFN-γ agonist therapy. Whether tumor growth decreases can be determined by measuring, e.g., actual tumor size, or any symptom associated with cancer such weight, fatigue, fever, changes in bowel or bladder function, and the like. Treatment or a reduction in tumor growth can also extend survival and improve quality of life.

The term “TNF agonist” refers to any compound or agent which induces or increases expression or production of TNF, directly binds to and increases the activity of TNF, mimics TNF binding to its receptor, or promotes binding of TNF to its receptor, TNFR1 and TNFR2. Thus, a “TNF agonist” may broadly be regarded as any compound or agent which agonizes or activates or promotes or stimulates or mimics TNF function. Suitable agonists may be TNF nucleic acids (i.e., nucleic acids that encode for TNF protein), TNF protein, TNF agonistic antibodies, or small molecule activators of TNF activity. A number of agents are known to agonize TNF including, e.g., lymphotoxin-α₂β that stimulates TNFR1 and TNFR2 signaling (Kucka, et al. (2021) Cell Death & Dis. 12:360); TNFR2 agonist antibodies (Ban, et al. (2008) Proc. Natl. Acad. Sci. USA 105(36):13644-13649; Melero, et al. (2013) Clin. Cancer Res. 19(5):1044-1053; WO 2016/110584 A1); oligomerized single chain TNF ligands (Fischer, et al. (2017) Scientific Reports 7:6607) and recombinant TNF-α protein (GENBANK Reference Sequence NP_000585.2) commercially available from sources such as R&D Systems, ThermoFisher Scientific and Peprotech. Any of the above agents, or combination of the above may be used as TNF agonists according to the present invention. Preferred TNF-α agonists are mammalian TNF-α protein, preferably human TNF-α protein and active fragments thereof.

The term “IFN-γ agonist” refers to any compound or agent which induces or increases expression or production of IFN-γ, directly binds to and increases the activity of IFN-γ, or promotes binding of IFN-γ to its receptor. Thus, a “IFN-γ agonist” may broadly be regarded as any compound or agent which agonizes or activates or promotes or stimulates or mimics IFN-γ function. Suitable agonists may be IFN-γ nucleic acids (i.e., nucleic acids that encode for IFN-γ protein), IFN-γ protein, IFN-γ agonistic antibodies, or small molecule activators of IFN-γ activity. A number of agents are known to agonize IFN-γ including, e.g., a C-terminal peptide of IFN-γ (i.e., IFN-γ (95-133; Szente, et al. (1996) J. Interferon Cytokine Res. 16(10):813-817); IFN-γ polypeptide variants (WO 2020/028275) and recombinant IFN-γ protein (GENBANK Reference Sequence NP_000610.2) commercially available from sources such as R&D Systems, ThermoFisher Scientific and Peprotech. Any of the above agents, or combination of the above may be used as IFN-γ agonists according to the present invention. Preferred IFN-γ agonists are mammalian IFN-γ protein, preferably human IFN-γ protein and active fragments thereof.

The present invention also provides methods for the manufacture of a medicament for treating a cancer and kits for carrying out the methods of treating cancer. The kits of the invention include at least one TNF agonist in an effective amount and at least one IFN-γ agonist in an effective amount. The kits may provide a single dose or multiple doses of TNF agonist(s) and IFN-γ agonist(s), wherein said agonists may be provided individually or in a co-formulation. The kit may take the form of a blister package; a lidded blister, a blister card or packet; a clamshell; an intravenous (IV) package, IV packette or IV container, a tray or a shrink wrap comprising the agonists and instructions for use of the composition for treating cancer.

### Administration of Inhibitors and Agonists

By “pharmaceutically acceptable” it is meant that the ingredients must be compatible with other ingredients of the composition as well as physiologically acceptable to the recipient. Pharmaceutically acceptable excipients such as vehicles, adjuvants, carriers or diluents are generally readily available. In addition, pharmaceutically acceptable auxiliary substances such as pH adjusting and buffering agents, isotonic agents, stabilizers, wetting agents and the like are generally readily available.

Pharmaceutical compositions may be formulated according to any of the conventional methods known in the art and widely described in the literature. Thus, the active ingredient may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions sterile packaged powders, and the like. In this respect, the pharmaceutical composition can be administered in various ways, for example, oral, buccal, rectal, parenteral, intraperitoneal, intradermal, subcutaneous, intramuscular, transdermal, intranasal, intrapulmonary, intratracheal, intratumoral, etc.

Examples of suitable carriers, excipients, and diluents are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methylhydroxyoenzoates, propyl hydroxybenzoates, talc, magnesium stearate, mineral oil or fatty substances such as hard fat or suitable mixtures thereof. The compositions may additionally include lubricating agents, wetting agents, emulsifying agents, suspending agents, preserving agents, sweetening agents, flavoring agents, and the like. The compositions of the invention may be formulated so as to provide quick, sustained or delayed release of the active ingredient after administration to the patient by employing procedures well known in the art.

Suitable doses will vary from patient to patient and can be determined by the physician in accordance with the weight, age and sex of the patient and the disease and also the particular inhibitors/agonists selected. A typical total daily dose may be in the range of 0.01 mg to 100 mg per kilogram body weight of an inhibitor/agonist, which may be administered as a single dose or in several smaller doses during the day. By way of illustration, typical dosing for golimumab, adalimumab, certolizumab and etanercept are 50 mg once a month, 40 mg every other week, 400 mg every two weeks and 50 mg once a week, respectively.

Subjects or patients suitable for treatment with a pharmaceutical composition or formulation may be identified by well-established indicators of the risk of developing the disease or by well-established characteristics of the disease present. For example, indicators of viral infection include fever, dry cough, shortness of breath (shortness of breath), headache, hypoxemia (low blood oxygen levels), lymphopenia (reduced lymphocyte count), and slightly elevated aminotransferase levels (liver damage). In certain aspects, subjects suitable for treatment exhibit the phenotype associated with COVID-19. indicators of cancer include, but are not limited to, fatigue, fever, changes in weight and a tumor.

Viral infections that can be treated with the therapy described herein include infections caused by or because of an arenavirus, coronavirus, filovirus, orthomyxovirus, paramyxovirus, or retrovirus family of viruses. In certain aspects, the viral infection is caused by or because of a virus selected from the group of Lassa Virus, Lymphocytic Choriomeningitis Virus (LCMV), Junin Virus, Machupo Virus, Guanarito Virus, Sabia Virus, Severe Acute Respiratory Syndrome (SARS) Virus, Murine Hepatitis Virus (MHV), Human Coronavirus, Bovine Coronavirus, Canine Coronavirus, Feline Infectious Peritonitis Virus, Ebola Virus, Marburg Virus, Influenza A Virus, Influenza B Virus, Influenza C Virus, Measles Virus, Mumps Virus, Canine Distemper Virus, Newcastle Disease Virus, Human Immunodeficiency Virus 1 (HIV-1), Human Immunodeficiency Virus 2 (HIV-2), Human T-cell Lymphotrophic Virus 1 (HTLV-1), Human T-cell Lymphotrophic Virus 2 (HTLV-2), Human Intracisternal A-type Particle 1 (HIAP-1), and Human Intracisternal A-type Particle 2 (HIAP-2). In certain aspects, the subject being treated has been diagnosed with a coronavirus, in particular an α-type human coronaviruses (HCoVs) such as HCoV-229E and HCoV-NL63; β-type HCoV-HKU1, SARS-CoV, MERS-CoV, and HCoV-OC43; or 2019-nCoV (i.e., SARS-CoV-2).

Bacterial infections that can be treated with the therapy described herein include infections caused by or because of Francisella tularensis, Burkholderia cenocepacia, Burkholderia multivorans, Staphylococcus aureus, Streptococcus pneumoniae, Mycobacterium tuberculosis, Bacillus anthracis, etc.

Fungal infections that can be treated with the therapy described herein include infections caused by or because of Aspergillus fumigatus, Candida albicans, Crytococcus neoformans, etc.

Parasitic infections that can be treated with the therapy described herein include infections caused by or because of Plasmodium sp., Toxoplasma gondii, etc.

Inflammatory diseases or conditions that can be treated with the therapy described herein include but are not limited to sepsis, hemophagocytic lymphohistiocytosis and TNF- and IFN-γ-associated cytokine shock and inflammation. As used herein, “TNF- and IFN-γ-associated cytokine shock and inflammation” refers to any inflammation mediated by the presence of TNF and IFN-γ and as such are disorders where inhibition of these cytokines and their signaling pathway would provide benefit. These disorders are likely to be regulated at least in part by the inflammatory cell death pathway activated by TNF and IFN-γ described herein. Diseases of particular note in this category include colitis, Crohn's disease, ulcerative colitis, IBD, HLH, psoriasis, idiopathic arthritic, uveitis, other autoinflammatory diseases, retinal detachment (and degeneration), retinitis pigmentosa, macular degeneration, pancreatitis, atopic dermatitis, arthritis (including rheumatoid arthritis, spondylarthritis, gout, systemic onset juvenile idiopathic arthritis (SoJIA), psoriatic arthritis), systemic lupus erythematosus (SLE), Sjogren's syndrome, systemic scleroderma, anti-phospholipid syndrome (APS), vasculitis, osteoarthritis, liver damage/diseases (non-alcohol steatohepatitis, alcohol steatohepatitis, autoimmune hepatitis, autoimmune hepatobiliary diseases, primary sclerosing cholangitis (PSC), acetaminophen toxicity, hepatotoxicity), kidney damage/injury (nephritis, renal transplant, surgery, administration of nephrotoxic drugs e.g., cisplatin, acute kidney injury (AKI)), Celiac disease, autoimmune idiopathic thrombocytopenic purpura (autoimmune ITP), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), neonatal hypoxic brain injury, allergic diseases (including asthma and atopic dermatitis), burns, multiple sclerosis, type I diabetes, Wegener's granulomatosis, pulmonary sarcoidosis, Behcet's disease, interleukin-1 converting enzyme (ICE, also known as caspase-1) associated fever syndrome (also known as periodic fever syndromes), chronic obstructive pulmonary disease (COPD), cigarette smoke-induced damage, cystic fibrosis, tumor necrosis factor receptor-associated periodic syndrome (TRAPS), a neoplastic tumor, periodontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, and rejection of transplant organs, tissues and cells.

Cancers that can be treated in accordance with the methods herein include, but are not limited to, acute lymphoblastic leukemia, acute myeloid leukemia, adrenocortical carcinoma, anal cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma (e.g., glioblastoma, astrocytoma, or oligodendrocytoma), hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplasia syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomyosarcoma, salivary gland cancer, sarcoma, skin cancer, small intestine cancer, soft tissue sarcoma, testicular cancer, throat cancer, thymic carcinoma, thyroid cancer, urethral cancer, uterine cancer, vaginal cancer, and Wilms tumor. In particular aspects, the methods of the invention are of use in the treatment of melanoma, lung cancer, colon cancer, or leukemia.

The following non-limiting examples are provided to further illustrate the present invention.

### Example 1: Antagonism of TNF and IFN-γ Blocks Inflammatory Cell Death

Mice. Irf1^{-/-} (Matsuyama, et al. (1993) Cell 75:83-97), Stat1^{-/-} (Durbin, et al. (1996) cell 84:443-450), Ripk3^{-/-} (Newton, et al. (2004) Mol. Cell. Biol. 24:1464-69), Ripk3^{-/-}Fadd^{-/-} (Dillon, et al. (2012) Cell Rep. 1:401-407), Ripk3^{-/-}Casp8^{-/-} (Oberst, et al. (2011) Nature 471:363-7), Apaf1^{-/-} (Jackson Laboratory) (Honarpour, et al. (2000) Dev. Biol. 218:248-258), Nos2^{-/-} (Jackson Laboratory) (Laubach, et al. (1995) Proc. Natl. Acad. Sci. USA 92:10688-92), Casp7^{-/-} (Lakhani, et al. (2006) Science 311:847-51), Casp3^{-/-} (Zheng, et al. (2000) Nat. Med. 6:1241-7), Gsdmd^{-/-} (Karki, et al. (2018) Cell 173:920-33 e913), Gsdme^{-/-} (Skarnes, et al. (2011) Nature 474:337-42), Mlk1^{-/-} (Murphy, et al. (2013) Immunity 39:443-53), Casp1^{-/-} (Man, et al. (2016) Cell 167:382-96 e317), Casp11^{-/-} (Kayagaki, et al. (2011) Nature 479:117-121), Casp1/11^{-/-} (Kayagaki, et al. (2011) Nature 479:117-121), Irf2^{-/-} (Kayagaki, et al. (2019) Sci. Signal 12), Irf3+(Sato, et al. (2000) Immunity 13:539-48), Irf7^{-/-} (Honda, et al. (2005) Nature 434:772-777), Irf3^{-/-}Irf7^{-/-} (Karki, et al. (2018) Cell 173:920-933 e913), Ifnar1^{-/-} (Muller, et al. (1994) Science 264:1918-21), Ifnar2^{-/-} (Fenner, et al. (2006) Nat. Immunol. 7:33-39), Irf9^{-/-} (Kimura, et al. (1996) Genes Cells 1:115-124), Trif^{-/-} (Yamamoto, et al. (2003) Science 301:640-43), Mda5^{-/-} (Kato, et al. (2006) Nature 441:101-105), Mavs^{-/-} (Suthar, et al. (2010) PLoS Pathog. 6:31000757), cGas^{-/-} (Schoggins, et al. (2014) Nature 505:691-5), Sting^{gt/gt} (Sauer, et al. (2011) Infect. Immun. 79:688-94), Gbp2^{-/-} (Degrandi, et al. (2013) Proc. Natl. Acad. Sci. USA 110:294-299), Ptpn6^{-/-} (Croker, et al. (2008) Proc. Natl. Acad. Sci. USA 105:15028-33) mice have been previously described. Irf5^{tm1Ppr}/J mice were purchased from The Jackson Laboratory and crossed with mice expressing Cre recombinase to generate Irf5^{-/-} mice. Gsdmd^{-/-}/Gsdme^{-/-} (Gsdmd/e^{-/-})Mlk1^{-/-} mice were generated by crossing Gsdme^{-/-}, Gsdmd^{-/-}, and Mlk1^{-/-} mice. Irf1^{-/-}Irf2^{-/-} mice were generated by crossing Irf1^{-/-} with Irf2^{-/-} mice. For SARS-CoV-2 infections, K18-ACE-2 transgenic mice were purchased from The Jackson Laboratory. All mice were generated on or extensively backcrossed to the C57/BL6 background.

All mice were bred at the Animal Resources Center at St. Jude Children's Research Hospital and maintained under specific pathogen-free conditions. Non-infectious animal studies were conducted under protocols approved by the St. Jude Children's Research Hospital committee on the Use and Care of Animals. SARS-CoV-2 infections were performed at the University of Tennessee Health Science Center under ABSL3 conditions using protocols approved by the UTHSC committee on the Use and Care of Animals.

Cell Culture and Stimulation Methods. Primary mouse bone marrow-derived macrophages (BMDMs) were generated from the bone marrow of wild-type and the indicated mutant mice. Cells were grown for 5-6 days in IMDM (Gibco) supplemented with 1% non-essential amino acids (Gibco), 10% FBS (Atlanta Biologicals), 30% L929 conditioned media, and 1% penicillin and streptomycin (Sigma). BMDMs were then seeded into antibiotic-free media at a concentration of 1×10⁶ cells into 12-well plates and incubated overnight. The human monocytic cell line THP-1 (ATCC) was cultured in RPMI media (Corning) supplemented with 10% FBS and 1% penicillin and streptomycin. The primary umbilical vein endothelial cells from normal human (HUVEC) (ATCC) were cultured in vascular cell basal medium (ATCC) containing cell growth factors (ATCC) and 1% penicillin and streptomycin. BMDMs were stimulated with the following cytokines where indicated unless otherwise noted: 20 ng/mL of IL-6 (Peprotech), 10 ng/mL of IL-18 (BioLegend), 20 ng/mL of IL-15 (R&D), 20 ng/mL of IL-1α (Peprotech), 20 ng/mL of IL-1β (R&D), 20 ng/mL of IL-2 (Peprotech), 25 ng/mL of TNF-α (Peprotech), 50 ng/mL of IFN-γ (Peprotech), 50 ng/mL of IFN-α (PBL Assay), 50 ng/mL of IFN-β (PBL Assay), or 50 ng/mL of IFN-h (Peprotech). For human cells, 50 ng/mL of TNF-α (Peprotech) and 100 ng/mL of IFN-γ (Peprotech) were used for the indicated time. For the inhibition of nitric oxide (NO), cells were co-treated with 1 mM of L-NAME hydrochloride (TOCRIS) or 100 μM of 1400W dihydrochloride (Enzo Life Sciences). SIN-1 chloride (TOCRIS) at the indicated concentrations was used as the NO donor.

Methods for Real-Time Imaging for Cell Death. The kinetics of cell death were determined using the INCUCYTE® S3 (Essen BioScience) live-cell automated system. BMDMs (5×10⁵ cells/well) were seeded in 24-well tissue culture plates. THP-1 (1×10⁵ cells/well) and HUVEC (5×10⁴ cells/well) were seeded in 48-well tissue culture plates. Cells were treated with the indicated cytokines and stained with propidium iodide (PI; Life Technologies) following the manufacturer's protocol. The plate was scanned, and fluorescent and phase-contrast images (4 image fields/well) were acquired in real-time every 1 hour from 0 to 48 hours post-treatment. PI-positive dead cells are marked with a red mask for visualization. The image analysis, masking, and quantification of dead cells were done using the software package supplied with the INCUCYTE® imager.

Immunoblot Analysis Methods. Cell lysates and culture supernatants were combined in caspase lysis buffer (containing protease inhibitors, phosphatase inhibitors, 10% NP-40, and 25 mM DTT) and sample loading buffer (containing SDS and 2-mercaptoethanol) for immunoblot analysis of caspases. For immunoblot analysis of signaling components, supernatants were removed, and cells were washed once with PBS, followed by lysis in RIPA buffer and sample loading buffer. Proteins were separated by electrophoresis through 8-12% polyacrylamide gels. Following electrophoretic transfer of proteins onto PVDF membranes (Millipore), nonspecific binding was blocked by incubation with 5% skim milk, then membranes were incubated with primary antibodies against: caspase-3 (Cell Signaling Technology [CST]), cleaved caspase-3 (CST), caspase-7 (CST), cleaved caspase-7 (CST), caspase-8 (AdipoGen), cleaved caspase-8 (CST), caspase-9 (CST), caspase-11 (Novus Biologicals), caspase-1 (AdipoGen), GAPDH (CST), iNOS (CST), pMLKL (CST), tMLKL (Abgent), pRIPK1 (CST), tRIPK1 (CST), GSDMD (Abcam), GSDME (Abcam), β-actin (Proteintech). Membranes were then washed and incubated with the appropriate horseradish peroxidase (HRP)-conjugated secondary antibodies (Jackson ImmunoResearch Laboratories, anti-rabbit, anti-mouse, or anti-rat). Proteins were visualized using Immobilon Forte Western HRP Substrate (Millipore).

Nitric Oxide Measurements. The amount of nitric oxide released in the supernatant of BMDMs stimulated with TNF-α alone, IFN-γ alone, or TNF-α+IFN-γ was determined using Griess reagent kit (Invitrogen) according to the manufacturer's instructions.

In vivo TNF-α and IFN-γ-Induced Inflammatory Shock. Six- to eight-week-old mice of the indicated genotypes were injected intraperitoneally with 10 μg TNF-α alone, 20 μg IFN-γ alone, or TNF-α+IFN-γ diluted in endotoxin-free PBS. Animals were under permanent observation, and survival was assessed every 30 minutes. Blood was collected 5 hours after cytokine injection. Blood composition was analyzed using an automated hematology analyzer. Serum lactate dehydrogenase (LDH), alanine aminotransferase (ALT), aspartate aminotransferase (AST), blood urea nitrogen (BUN), and ferritin were analyzed by colorimetry using respective kits (LDH, ALT, AST, and BUN, all from HORIBA; and ferritin from Abcam) according to the manufacturer's instructions.

Flow Cytometry Methods. Peripheral blood was mixed with ACK lysis buffer for 3 minutes to lyse the red blood cells (RBCs). The cells were then stained with monoclonal antibodies for the following markers for flow cytometry: CD11b from eBioscience; F4/80, Ly6C, and Ly6G from BioLegend; and CD19, TCRb, and CD45.2 from Tonbo Biosciences. Cells were gated on live single-cell populations and hematopoietic cells using the CD45.2 gate followed by separation of each of the specific cell populations using the following cell surface markers: macrophages (CD11b+, F4/80+), neutrophils (CD11b+, Ly6Clow, Ly6Ghigh), T cells (TCRb+, CD19−), and B cells (CD19+, TCRb−). 40

Histopathology Methods. Lungs and intestines were fixed in 10% formalin, then processed and embedded in paraffin by standard procedures. Sections (5 μM) were stained with hematoxylin and eosin (H&E) and examined by a pathologist blinded to the experimental groups. For immunohistochemistry, formalin-fixed paraffin-embedded lungs and intestines were cut into 4 μM sections. Cleaved caspase-3 (Essen Bioscience) and CD45 (BD Pharmingen) staining was performed according to the manufacturer's instructions. TUNEL (terminal deoxynucleotidyl transferase deoxyuridine triphosphate nick-end labeling) staining was performed using the Dead-End™ kit (Promega) according to the manufacturer's instructions. The number of cleaved caspase-3- and TUNEL-positive cells in five high power fields (20×) were counted per mouse.

COVID-19 Patient Cytokine Analysis. COVID-19 patient cytokine data was obtained from the scientific literature (Lucas, et al. (2020) Nature 584:463-9). The list of the top 10 differentially present pro-inflammatory cytokines was manually generated. Amounts of selected cytokines were averaged for healthy control subjects, and moderate and severe COVID-19 patients. Heatmaps were generated using Morpheus (Broad Institute). Cytokine data for the serum levels of TNF-α and IFN-γ were from the literature (Silvin, et al. (2020) Cell 182:1401-18 e1418) for healthy patients and patients with moderate, severe, and critical COVID-19.

Gene Expression Analysis. Genes involved in type II interferon signaling were selected based on annotations (Hadjadj, et al. (2020) Science 369:718-24). Nanostring nCounter data were obtained for healthy patients and patients with moderate, severe, and critical COVID-19. Heatmaps were generated based on average expression of selected genes using Morpheus. NOS2 expression in COVID-19 patients was extracted from the same Nanostring nCounter data (Hadjadj, et al. (2020) Science 369:718-24). Murine orthologs of the genes identified as the most differentially regulated in humans were manually selected from microarray-based transcriptomics analysis of BMDMs treated with TNF-α and IFN-γ and used to generate heatmaps using Morpheus. Additionally, NF-κB target genes for both inflammatory cytokines and apoptosis regulators were selected from the microarray of BMDMs.

Single-Cell RNA-Seq Data Analysis. The feature-barcode matrix was downloaded from GSE149689 (Lee, et al. (2020) Sci. Immunol. 5(49):eabd1554). Low quality cells were excluded from the analysis if the mitochondrial genes represented >15% or if the number of features in a cell was <200. Count values were normalized, log-transformed, and scaled using the Seurat R package v3.1.4 (Satija, et al. (2015) Nat. Biotechnol. 33:495-502). The top 15 dimensions and a resolution value of 0.5 were used for UMAP dimension reduction (Lee, et al. (2020) Sci. Immunol. 5(49):eabd1554).

SARS-CoV-2 Culture Methods. The SARS-CoV-2 isolate USA-WA1/2020 was obtained through BEI Resources (NIAID, NIH: SARS-Related Coronavirus 2, Isolate USA-WA1/2020) and amplified in Vero-E6 cells (ATCC) at an MOI of 0.1 in Minimal Essential Medium (MEM; Corning) supplemented with 5% heat-inactivated FBS (Gibco) and 1% L-Glutamine (Corning) and 5 mM penicillin/streptomycin (Gibco). Following virus amplifications, viral titer was determined using a plaque assay using the method described previously for alphaviruses (Lee, et al. (2020) J. Virol.). All experiments involving SARS-CoV-2 were done in a biosafety level 3 laboratory.

In vitro Infection of PBMCs Methods. Blood was collected from anonymous healthy donors at St. Jude Children's Research Hospital following IRB-approved protocols. Human PBMCs were isolated from the blood by density gradient using colloidal silica coated with polyvinylpyrrolidone sold under the tradename PERCOLL® (GE Healthcare). PBMCs were washed and resuspended in RPMI 1640 supplemented with 10% FBS. For SARS-CoV-2 infection, 1×10⁶ PBMCs from 3 healthy donors were seeded into 12-well plates and infected with indicated multiplicity of infection (MOI) of the virus. Supernatants from medium-treated or infected PBMCs were collected at 24 hours post-infection. The pro-inflammatory cytokines in the supernatant were analyzed by multiplex ELISA (Millipore) according to the manufacturer's instructions.

SARS-CoV-2 Infection in Mice. Age- and gender-matched, 7- to 8-week old K18-ACE-2 transgenic mice were anesthetized with 250 mg/kg Avertin and then infected intranasally with SARS-CoV-2 in 50 μl PBS containing approximately 2×10⁴ PFU. Infected mice were administered intraperitoneally 200 μl of PBS containing no antibody, 500 μg of isotype control (Leinco Technologies, Inc.), or 500 μg of neutralizing antibody against TNF-α (Leinco Technologies, Inc.) plus 500 μg of neutralizing antibody against IFN-γ (Leinco Technologies, Inc.) on days 1, 3, and 4 post-infection. Mice were monitored over a period of 14 days for survival.

LPS-Induced Sepsis Methods. Age- and gender-matched, 7- to 8-week old wild-type mice were injected intraperitoneally with 20 mg/kg body weight of lipopolysaccharide (LPS; Sigma). Mice were administered intraperitoneally with 200 μl of PBS containing no antibody, 500 μg of isotype control, 500 μg of neutralizing antibody against TNF-α, 500 μg of neutralizing antibody against IFN-γ, or 500 μg each of neutralizing antibodies against TNF-α and IFN-γ 30 minutes and 6 hours post-LPS injection. Mice were monitored over a period of 3 days for survival.

Poly I:C and LPS-Induced Hemophagocytic Lymphohistiocytosis (HLH) Methods. HLH was induced by sequential challenge of poly I:C and LPS as described previously (Wang, et al. (2019) Proc. Natl. Acad. Sci. USA 116:220-2209). Age- and gender-matched, 7- to 8-week old wild-type mice were injected intraperitoneally with 10 mg/kg body weight of high molecular weight poly I:C (tlrl-pic, InvivoGen). Mice were then administered intraperitoneally 200 μl of PBS containing no antibody, 500 μg of isotype control, 500 μg of neutralizing antibody against TNF-α, 500 μg of neutralizing antibody against IFN-γ, or 500 μg each of neutralizing antibodies against TNF-α and IFN-γ 24 hours post-poly I:C injection. All the mice were then challenged with a sub-lethal dose of LPS (5 mg/kg body weight) 1 hour after the above-mentioned treatments. Mice were monitored over a period of up to 3 days for survival.

Statistical Analysis. GraphPad Prism 8.0 software was used for data analysis. Data are shown as mean±SEM. Statistical significance was determined by t tests (two-tailed) for two groups or one-way ANOVA or two-way ANOVA for three or more groups. Survival curves were compared using the log-rank (Mantel-Cox) test. P<0.05 was considered statistically significant.

Inflammatory Cytokines are Elevated in Patients with COVID-19, and Synergy Between TNF and IFN-γ Specifically Induces Cell Death. Studies of patients with COVID-19 have reported associations between disease severity and an influx of innate immune cells and inflammatory cytokines. To determine the pro-inflammatory cytokines that are the most highly upregulated during SARS-CoV-2 infection, a publicly available dataset was re-analyzed for circulating cytokines from healthy volunteers and patients with moderate or severe COVID-19. It was observed that levels of circulating IL-6, IL-18, IFN-γ, IL-15, TNF-α, IL-1α, IL-1β, and IL-2 were the most upregulated in patients with moderate or severe COVID-19. This is consistent with a recent study, which found that infection with MHV, a murine coronavirus, induces the release of IL-6, TNF, IL-18, and IL-1 (Zheng, et al. (2020) J. Biol. Chem. 295:14040-14052). Several other studies have also demonstrated the increased production of these subsets of pro-inflammatory cytokines in patients with COVID-19 (Huang, et al. (2020) Lancet 395:497-506; Blanco-Melo, et al. (2020) Cell 181:1036-45 e1039; Del Valle, et al. (2020) Nat. Med. 26:1636-43). Additionally, SARS-CoV-2-infected peripheral blood mononuclear cells (PBMCs) obtained from healthy donors showed increased production of pro-inflammatory cytokines. A direct correlation between systemic increases in pro-inflammatory cytokines and lung injury, multiple organ failure, and unfavorable prognosis in patients with severe COVID-19 has been suggested, and uncontrolled cytokine signaling can contribute to multi-organ damage by inducing cell death (Jose & Manuel (2020) Lancet Respir. Med. 8:e46-e47; Mehta, et al. (2020) Lancet 395:1033-1034; Ragab, et al. (2020) Front. Immunol 11:1446). To understand the ability of the pro-inflammatory cytokines released during SARS-CoV-2 infection to induce cell death, bone marrow-derived macrophages (BMDMs) were treated with the cytokines that were the most highly upregulated in the circulation of patients with COVID-19 and in PBMCs infected with SARS-CoV-2. None of the cytokines individually induced high levels of cell death in BMDMs at the concentration used. However, the combination of all these cytokines (IL-6, IL-18, IFN-γ, IL-15, TNF-α, IL-1α, IL-1β, and IL-2; Cocktail-1) robustly induced cell death, suggesting that synergistic cytokine signaling is required for this process. To determine the specific cytokines involved in this synergy, all possible combinations of two cytokines were prepared. Out of the 28 combinations tested, only the combination of TNF-α and IFN-γ induced cell death to a similar extent as the cytokine cocktail (Cocktail-1). Treating with Cocktail-2, which lacked TNF-α and IFN-γ, failed to induce similar levels of cell death. TNF-α or IFN-γ may synergize with Cocktail-2 to induce cell death. Similarly, addition of either TNF-α or IFN-γ to Cocktail-2 still failed to induce cell death, further supporting that synergism between TNF-α or IFN-γ is critical in inducing cell death. Subsequently, it was evaluated whether IFN-γ could be replaced with other IFNs that engage type I or type III IFN pathways, which are also important for immune regulation. Combining TNF-α with IFN-α, IFN-β, or IFN-A failed to induce high levels of cell death, indicating that type II IFN signaling specifically co-ordinates with TNF signaling to induce cell death.

The dynamics of cell death could be affected by the concentration of cytokines chosen. To confirm that the observed effect was not an artifact of the concentration chosen, it was observed that increasing the concentration of cytokines in Cocktail-2 up to 10-fold still failed to induce cell death. Conversely, the kinetics of cell death induced by TNF-α and IFN-γ co-stimulation was proportional to the concentration of TNF-α and IFN-γ. Similar to the cell death observed in murine BMDMs, TNF-α and IFN-γ treatment also induced robust cell death in the human monocytic cell line THP-1, and primary human umbilical vein endothelial cells (HUVEC).

To understand the timing of TNF-α and IFN-γ release during the disease course of COVID-19, another public dataset was analyzed (Silvin, et al. (2020) Cell 182:1401-18 e1418). This analysis indicated that increased production of TNF-α occurs in patients with moderate COVID-19, while production of IFN-γ occurs in patients with severe COVID-19. This indicates that production of TNF-α precedes IFN-γ in COVID-19. Additionally, the increased production of TNF-α and IFN-γ observed in patients with COVID-19 can be contributed by multiple cell types. To determine the contribution of immune cells in the production of TNF-α and IFN-γ, publicly available single-cell RNA-seq data of PBMCs obtained from healthy donors and patients with mild or severe COVID-19 (Lee, et al. (2020) Sci. Immunol. 5(49):eabd1554) were re-analyzed. While macrophages from the PBMCs of patients with COVID-19 showed increased expression of TNF-α and IL-1α, NK cells and CD8+ T cells had increased expression of IFN-γ compared with healthy donors.

TNF and IFN-γ Shock Mirrors COVID-19 Symptoms. Patients with COVID-19 who require ICU supportive care often present with ARDS, acute cardiac injury, and acute kidney injury (Pan, et al. (2020) Lancet Respir. Med. 8:816-821). These symptoms can be fatal and have been associated with cytokine storm. To examine whether TNF-α and IFN-γ can induce COVID-19-related symptoms, a murine model of TNF-α and IFN-γ shock was developed. Similar to in vitro data, where treatment with TNF-α or IFN-γ alone failed to induce cell death, administration of TNF-α or IFN-γ did not cause significant mortality in mice. However, treating with the combination of TNF-α and IFN-γ led to synergistic mortality, indicating that the TNF-α and IFN-γ-mediated cell death may be associated with mortality. It was subsequently determined which cell types and organs were affected by the TNF-α and IFN-γ shock. This analysis indicated an increased influx of inflammatory cells in the lamina propria of the intestine of mice treated with TNF-α and IFN-γ compared with PBS-treated mice. Similarly, lungs from TNF-α and IFN-γ-treated mice showed septal thickening due to the accumulation of neutrophils in capillaries. Also, the incidence of caspase-3- and TUNEL-positive intestinal crypts and caspase-3-positive lung cells was increased in TNF-α and IFN-γ-treated mice compared with PBS-treated mice, indicating that TNF-α and IFN-γ induce lung and intestinal damage and cell death. The increased cell death in the TNF-α and IFN-γ-treated mice was further confirmed by the presence of elevated serum LDH levels.

It was next determined whether TNF-α and IFN-γ shock could mimic the laboratory abnormalities observed in patients with COVID-19. In patients who succumb to COVID-19, the level of ALT, AST, blood urea nitrogen (BUN), and ferritin have been shown to increase until death (Ghahramani, et al. (2020) Eur. J. Med. Res. 25:30; Huang, et al. (2020) Lancet 395:497-506). Similarly, increased ALT, AST, BUN, and ferritin was observed in the serum of mice subjected to TNF-α and IFN-γ shock. Additionally, a meta-analysis of nine studies of patients with COVID-19 showed that thrombocytopenia is associated with a five-fold increased risk of severity and mortality (Lippi, et al. (2020) Clin. Chim. Acta 506:145-148). In the murine model, complete blood counts (CBC) revealed a decrease in the number of thrombocytes and percentage of plateletcrit in the blood of mice co-treated with TNF-α and IFN-γ compared with the PBS-treated group. Although alterations in RBC count and hematocrit percentage have not been reported in patients with COVID-19, increased RBC count, hematocrit percentage, and hemoglobin levels were observed in the blood of mice subjected to TNF-α and IFN-γ treatment.

Lymphopenia is another one of the hallmarks of severity and hospitalization in patients with COVID-19 (Tan, et al. (2020) Signal Transduct. Target Ther. 5:33; Zhao, et al. (2020) Int. J. Infect. Dis. 96:131-135). A meta-analysis revealed an increased neutrophil-to-lymphocyte ratio (NLR) in patients with severe COVID-19 compared with patients with nonsevere disease (Chan & Rout (2020) J. Clin. Med. Res. 12:448-453). In the murine model of TNF-α and IFN-γ shock, immune profiling of the peripheral blood identified an overall increase in the presence of innate immune cell lineages including macrophages and neutrophils with a reduction in the percentage of B cells and T cells, leading to a concomitant increase in NLR in TNF-α and IFN-γ-treated mice compared with PBS-treated mice. Overall, these data indicate that TNF-α and IFN-γ cytokine shock in mice mirrors the symptoms of COVID-19 and that these cytokines could be the specific factors underlying the pathophysiology of this pandemic disease.

TNF and IFN-γ Induce Pyroptosis, Apoptosis, and Necroptosis (PANoptosis). Studies have linked cytokine storm to lung injury, multiple organ failure, and poor prognosis for patients with COVID-19 (Jose & Manuel (2020) Lancet Respir. Med. 8:e46-e47; Mehta, et al. (2020) Lancet 395:1033-1034). Cytokine storm may cause this organ damage through inflammatory cell death, a lytic form of death that is associated with the secretion of cytokines that trigger inflammation and the release of other factors that alert immune cells of imminent danger. In contrast, classical apoptosis was thought to avoid strong immune stimulation by allowing the cellular content to be taken up and recycled by other cells during the cell death process. Although apoptosis has long been regarded as a non-inflammatory mode of cell death, recent studies suggest that this is not always the case. As the cell death field has advanced, two inflammatory cell death pathways, pyroptosis and necroptosis, have been defined. Furthermore, the apoptotic caspase-8 can mediate cleavage of GSDMD to induce pyroptosis. Therefore, thoroughly defining the nature of cell death induced by the combination of TNF-α and IFN-γ, which had previously been reported to induce immunologically silent apoptosis (Selleri, et al. (1995) J. Cell Physiol. 165:538-546), and using genetic evidence to dissect the signaling pathway activated and the molecular mechanisms involved is critical to identify approaches for specifically targeting these molecules in therapy.

Pyroptosis. To investigate whether TNF-α and IFN-γ together can induce inflammatory cell death in the form of pyroptosis, the cleavage of GSDMD was monitored. Co-treatment of TNF-α and IFN-γ induced a small amount of cleavage of GSDMD to produce the active P30 fragment that can form membrane pores to induce pyroptosis. GSDMD can be processed to release this P30 fragment by caspase-1, downstream of inflammasome activation, or caspase-11 (He, et al. (2015) Cell Res. 25:1285-98; Kayagaki, et al. (2015) Nature 526:666-671; Shi, et al. (2015) Nature 526:660-665). Consistent with the small amount of GSDMD P30 production, there was no activation of caspase-1 and minimal cleavage of caspase-11. Another member of the gasdermin family, GSDME, has also been shown to induce pyroptosis under specific conditions (Wang, et al. (2017) Nature 547:99-103). This analysis indicated that BMDMs stimulated with the combination of TNF-α and IFN-γ displayed robust cleavage of pyroptotic GSDME, demonstrating that TNF-α and IFN-γ together induced pyroptosis in BMDMs.

Apoptosis. In addition to pyroptosis, it was also found that TNF-α and IFN-γ induced apoptosis as evidenced by the cleavage of apoptotic caspases caspase-3, -7, -8, and -9 in BMDMs treated with TNF-α and IFN-γ. Furthermore, recent studies have shown that activation of caspase-3 and -7 can inactivate GSDMD by processing it to produce a P20 fragment (Chen, et al. (2019) EMBO J. 38:e101638; Taabazuing, et al. (2017) Cell. Chem. Biol. 24:507-514 e504), a result that was also observed in the present analysis.

Necroptosis. It was also determined whether the combination of TNF-α and IFN-γ induced necroptosis. Cells stimulated with TNF-α and IFN-γ showed robust phosphorylation of MLKL, indicating that necroptosis is occurring. During necroptosis, phosphorylation of MLKL occurs downstream of activation of the protein kinases RIPK1 and RIPK3. During TNF-α and IFN-γ treatment, phosphorylation of RIPK1 was observed. In addition, cleavage of total RIPK1 was seen, a feature which has been previously shown to be involved in regulating apoptosis and necroptosis (Newton, et al. (2019) Nature 574:428-431).

To investigate the relative contribution of TNF-α or IFN-γ in activating these cell death pathways, BMDMs were stimulated with TNF-α alone, IFN-γ alone, or the combination of TNF-α and IFN-γ. TNF-α or IFN-γ alone did not induce robust cleavage of GSDMD and GSDME. In addition, reduced cleavage of caspase-3, -7 and -8 was observed in cells stimulated with TNF-α or IFN-γ compared with the combination. Furthermore, TNF-α or IFN-γ failed to induce robust MLKL phosphorylation. However, the combination of TNF-α and IFN-γ synergistically induced cleavage of GSDME, caspase-3, -7, and -8, and phosphorylation of MLKL. Collectively, these data indicate that TNF-α and IFN-γ together sensitize the cells to undergo inflammatory cell death involving the components of pyroptosis, apoptosis, and necroptosis, showing the PANoptosis is occurring.

Signal Transducer and Activator of Transcription 1 (STAT1)/Interferon Regulatory Factor 1 (IRF1) is Required for Nitric Oxide Production and Subsequent PANoptosis Induced by TNF and IFN-γ. The best-described signaling pathways for TNF-α and IFN-γ are NF-κB and IFN signaling pathways, respectively; however, these pathways have counteracting effects. While NF-kB activation generally drives pro-survival signaling (Liu, et al. (2017) Signal Transduct. Target Ther. 2; Papa, et al. (2004) J. Cell Sci. 117:5197-5208), IFN-γ-mediated signaling is cytotoxic (Lin et al., 2017). To understand how these signaling pathways were affected by the combination of TNF-α and IFN-γ, microarray analysis was performed to identify the most highly upregulated type II IFN-responsive genes in wild-type BMDMs co-treated with TNF-α and IFN-γ. In parallel, to determine which genes had relevance in human patients, a publicly available dataset was re-analyzed for differentially regulated type II IFN-responsive genes in patients with differing severities of COVID-19. These analyses indicated that the genes encoding IRF1, IRF5, IRF7, JAK2, and PML were highly upregulated in both patients with severe COVID-19 and TNF-α and IFN-γ-treated BMDMs. To investigate the role of IRF1, IRF5, and IRF7 in instigating inflammatory cell death in response to TNF-α and IFN-γ, Irf1^{-/-}, Irf5^{-/-} and Irf7^{-/-} BMDMs were analyzed. Cells deficient in IRF1, but not IRF5 or IRF7, were protected from cell death upon treatment with TNF-α and IFN-γ.

JAK2 is known to signal upstream of IRF1 through STAT1; autophosphorylation of JAK2 phosphorylates JAK1 to activate the transcription factor STAT1, which localizes to the nucleus to induce transcription of type II IFN-responsive genes, including IRF1 (Schroder, et al. (2004) J. Leukoc. Biol. 75:163-189). Based on the upregulation of JAK2 in both patients with severe COVID-19 and BMDMs treated with TNF-α and IFN-γ and based on the confirmed role of IRF1 in promoting cell death in this pathway, the role of upstream molecules in TNF-α and IFN-γ-mediated cell death was also assessed. Similar to IRF1-deficient cells, BMDMs lacking STAT1 were protected from cell death. Consistent with this protection, Irf1^{-/-} BMDMs showed impaired activation of apoptotic caspases (caspase-3, -7, and -8); the pyroptotic molecule GSDME; and the necroptotic molecule MLKL. It is possible that type II IFN-responsive genes other than IRF1 that were induced in patients with severe COVID-19 or TNF-α and IFN-γ co-treated BMDMs contributed to the cell death. To determine whether these other molecules were contributing, cell death in cells lacking molecules involved in IFN production or signaling including IRF1, IRF2, IRF3, IRF5, IRF7, IFNAR1, IFNAR2, IRF9, STAT1, TRIF, MDA5, MAVS, cGAS, STING, GBP2, and PTPN6 were analyzed. These analyses indicated that TNF-α and IFN-γ co-treatment-induced cell death was only impaired in IRF1 and STAT1-deficient BMDMs. Altogether, these findings indicate that the STAT1/IRF1 axis regulates inflammatory cell death in response to TNF-α and IFN-γ treatment.

Given that IRF1 is a transcription factor, it is likely that it regulates inflammatory cell death by inducing type II IFN-responsive genes. Using a microarray, genes were identified which had the lowest levels of expression in Irf1^{-/-} co-stimulated with TNF-α and IFN-γ compared with the similarly stimulated wild-type BMDMs. Nos2, the gene encoding iNOS, was found to be one of the most downregulated genes in Irf1^{-/-} BMDMs under these conditions. The protein levels of iNOS were also reduced in Irf1^{-/-} cells treated with TNF-α and IFN-γ. In addition, iNOS protein expression was abolished in Stat1^{-/-} cells during treatment with TNF-α and IFN-γ. Consistent with the reduced iNOS expression, reduced production of nitric oxide (NO) in Irf1^{-/-} and Stat1^{-/-} cells was observed compared with wild-type cells upon treatment with TNF-α and IFN-γ. Together, these results indicate that iNOS is an IFN-inducible protein which is produced through a mechanism requiring STAT1 and IRF1 downstream of TNF-α and IFN-γ stimulation.

To determine whether iNOS had any impact in COVID-19 pathogenesis, a publicly available dataset was analyzed. This analysis indicated that NOS2 is significantly upregulated in patients with severe and critical COVID-19 compared with healthy controls. iNOS and the downstream NO are involved in numerous biological processes (Xu, et al. (2002) Cell. Res. 12:311-320), and NO can be cytotoxic or cytostatic depending upon the context (Albina & Reichner (1998) Cancer Metastasis Rev. 17:39-53; Pervin, et al. (2001) Proc. Natl. Acad. Sci. USA 98:3583-88). To investigate whether iNOS and NO play important roles in the cell death induced by TNF-α and IFN-γ treatment, cell death was investigated in Nos2^{-/-} BMDMs. Upon treatment with TNF-α and IFN-γ, Nos2^{-/-} BMDMs were protected from cell death. Wild-type BMDMs were also treated with NO production inhibitors and it was found that both L-NAME and 1400W inhibited cell death in response to TNF-α and IFN-γ treatment. Consistent with the impaired cell death, Nos2^{-/-} BMDMs had reduced activation of apoptotic caspases (caspase-3, -7, and -8), the pyroptotic molecule GSDME, and the necroptotic molecule MLKL. Overall, these data indicate that STAT1/IRF1 regulates iNOS expression for the production of NO, which subsequently induces inflammatory cell death in response to TNF-α and IFN-γ co-treatment.

While TNF-α or IFN-γ alone have been shown to induce NO (Salim, et al. (2016) PLoS One 11:e0153289), they each failed to induce cell death on their own. Moreover, NO has been demonstrated to be cytotoxic or cytostatic depending upon the context (Albina & Reichner (1998) Cancer Metastasis Rev. 17:39-53; Pervin, et al. (2001) Proc. Natl. Acad. Sci. USA 98:3583-88). Therefore, it is possible that the concentration of NO is critical to induce cell death and that synergism of TNF-α and IFN-γ produces the increased concentration of NO necessary to activate cell death pathways. Indeed, TNF-α and IFN-γ synergistically induced iNOS and produced more NO than TNF-α or IFN-γ alone. To further confirm that the cytotoxicity of NO was concentration-dependent, wild-type BMDMs were treated with increasing concentrations of the NO donor SIN-1. This analysis indicated that the kinetics of cell death induced by SIN-1 were proportional to the NO concentration. Altogether, these data indicate that TNF-α and IFN-γ synergistically induce iNOS and NO, which subsequently induce cell death.

However, it is possible that cell death in response to TNF-α and IFN-γ may be induced through another route; IFN-γ may interfere with TNF-α signaling to suppress NF-κB activation and switch the pro-survival signaling of TNF-α to cell death. TNF-α signaling engages RIPK1 to induce caspase-8-driven apoptosis or RIPK3-driven necroptosis in the context of NF-κB inhibition or caspase-8 inhibition, respectively (Lin, et al. (1999) Genes Dev. 13:2514-26; Vercammen, et al. (1998) J. Exp. Med. 187:1477-1485). To address the possibility that IFN-γ is suppressing NF-κB to drive TNF-α-induced cell death, the microarray data were re-analyzed to assess the expression of NF-κB target genes for inflammatory cytokines and apoptosis regulators in wild-type BMDMs treated with TNF-α alone or the combination of TNF-α and IFN-γ. This analysis indicated that the expression NF-κB target genes for inflammatory cytokines was not impaired in the cells stimulated with TNF-α and IFN-γ compared with their expression in cells stimulated with TNF-α alone. Moreover, treatment with IFN-γ did not influence TNF-α-driven expression of genes that regulate apoptosis. These data indicate that IFN-γ does not suppress TNF-α-induced NF-kB signaling to induce cell death. Collectively, these data show that the cell death induced by TNF-α and IFN-γ is driven by the IRF1/STAT1 pathway and involves the induction of iNOS and NO, which subsequently cause cell death.

RIPK1/Fas Associated Death Domain (FADD)/CASP8 Axis Drives PANoptosis Induced by TNF and IFN-γ. NO is known to induce apoptosis by activating caspase-8-dependent pathways (Du, et al. (2006) Am. J. Physiol. Renal. Physiol. 290:F1044-1054; Dubey, et al. (2016) Cell Death Dis. 7:e2348). In addition to its classical role in inducing apoptosis, caspase-8 plays a critical role in regulating inflammatory cell death pathways, pyroptosis and necroptosis (Fritsch, et al. (2019) Nature 575:683-7; Kuriakose, et al. (2016) Sci. Immunol. 1; Newton, et al. (2019) Nature 574:428-431; Orning, et al. (2018) Science 362:1064-1069; Sarhan, et al. (2018) Proc. Natl. Acad. Sci. USA 115:E10888-E10897). To determine whether caspase-8 mediates the cell death induced by TNF-α and IFN-γ, BMDMs derived from Ripk3^{-/-} and Ripk3^{-/-} Casp8^{-/-} mice were analyzed, since caspase-8-deficient mice are not viable. RIPK3 deficiency failed to protect against cell death, whereas deletion of both RIPK3 and caspase-8 provided substantial protection against the cell death induced by TNF-α and IFN-γ cotreatment. The importance of caspase-8 in inflammatory cell death in response to TNF-α and IFN-γ was further supported by impaired activation of apoptotic caspases (caspase-3, -7, and -9); the pyroptotic molecule GSDME; and the necroptotic molecule MLKL in Ripk3^{-/-} Casp8^{-/-} cells compared with their activation in wild-type cells.

Caspase-8 is recruited to cell death-inducing complexes by FADD. To address whether FADD regulates pyroptosis, apoptosis, and necroptosis induced by TNF-α and IFN-γ co-treatment, BMDMs derived from Ripk3^{-/-} and Ripk3^{-/-} Fadd^{-/-} mice were examined, since FADD-deficient mice are not viable. As before, loss of RIPK3 did not protect cells from undergoing death; however, deletion of both RIPK3 and FADD provided substantial protection against the cell death induced by TNF-α and IFN-γ co-treatment. Reduced activation of caspase-3, -7, -8, and -9; GSDME; and MLKL was also observed in Ripk3^{-/-}Fadd^{-/-} cells compared with wild-type cells. These data indicate that caspase-8 and FADD are key regulators of pyroptosis, apoptosis, and necroptosis in response to TNF-α and IFN-γ.

APAF1/caspase-9 are known to be mediators of the intrinsic apoptotic pathway. To study the role of intrinsic apoptosis in response to TNF-α and IFN-γ stimulation, BMDMs deficient in APAF1 were used. Similar dynamics of cell death between wild-type and Apaf1^{-/-} BMDMs were observed, indicating that intrinsic apoptosis does not regulate the cell death triggered by TNF-α and IFN-γ co-treatment in macrophages. Collectively, these data indicate that the FADD/caspase-8 axis regulates TNF-α and IFN-γ co-treatment-induced inflammatory cell death independent of intrinsic apoptosis in macrophages.

Subsequently, the contribution of downstream molecules activated by caspase-8 in TNF-α and IFN-γ-induced cell death was examined. BMDMs deficient in caspase-3, but not caspase-7, showed reduced cell death with respect to wild-type cells. Loss of the necroptotic executioner MLKL or the pyroptotic executioner GSDMD failed to protect cells against death triggered by TNF-α and IFN-γ co-treatment. In addition, loss of the upstream activators of GSDMD, caspase-1 and caspase-11, did not protect against cell death. Similar to caspase-3-deficient cells, cells lacking GSDME showed reduced cell death compared with wild-type cells in response to TNF-α and IFN-γ co-treatment. However, deletion of GSDME with the other pore-forming molecules MLKL and GSDMD showed a similar level of protection as that of cells lacking GSDME alone, indicating that GSDME and caspase-3 potentiate the cell death in response to TNF-α and IFN-γ co-treatment.

Inhibition of Cell Death Provides Protection Against Lethality Induced by TNF-α and IFN-γ. Based on the findings that the STAT1/IRF1/caspase-8 axis drives inflammatory cell death downstream of TNF-α and IFN-γ, it was hypothesized that inhibition of this pathway would provide protection against TNF-α and IFN-γ-induced shock in vivo. Indeed, mice deficient in STAT1 or both RIPK3 and caspase-8 were resistant to mortality induced by the cytokine shock, while mice deficient in RIPK3 alone were not, indicating that STAT1-mediated, caspase-8-dependent cell death contributes to the fatal outcome.

Next, it was investigated whether deletion of STAT1 or caspase-8 would rescue the laboratory abnormalities caused by TNF-α and IFN-γ shock that are consistent with the symptoms observed in patients with COVID-19. There was increased cell death as measured by serum LDH in wild-type mice subjected to TNF-α and IFN-γ shock compared with PBS-treated wild-type mice. Consistent with the reduced cell death observed in vitro, the LDH level of TNF-α and IFN-γ-treated Stat1^{-/-} or Ripk3^{-/-}Casp8^{-/-} mice was similar to PBS-treated Stat1^{-/-}, Ripk3^{-/-}Casp8^{-/-}, or wild-type mice, indicating that STAT1^{-/-} and caspase-8-mediated cell death is driving lethality in mice subjected to TNF-α and IFN-γ shock. Similarly, deletion of STAT1 or caspase-8 reduced the level of ALT and AST in mice treated with TNF-α and IFN-γ shock. However, Stat1^{-/-} and Ripk3^{-/-}Casp8^{-/-} mice co-treated with TNF-α and IFN-γ had a decreased percentage of T cells in the blood when compared to their PBS-treated counterparts. The number of thrombocytes and percentage of plateletcrit in the blood of Stat1^{-/-} or Ripk3^{-/-}Casp8^{-/-} mice co-treated with TNF-α and IFN-γ were generally similar to those of wild-type mice treated with PBS. Moreover, RBC count, hematocrit percentage, and hemoglobin levels in the blood of TNF-α and IFN-γ-treated Stat1+ or Ripk3^{-/-} Casp8^{-/-} mice were similar to PBS-treated Stat1^{-/-}, Ripk3^{-/-}Casp8^{-/-}, or wild-type mice. Altogether, these data indicate that inhibition of inflammatory cell death driven by the STAT1/caspase-8 axis is crucial to prevent TNF-α and IFN-γ-mediated mortality in vivo.

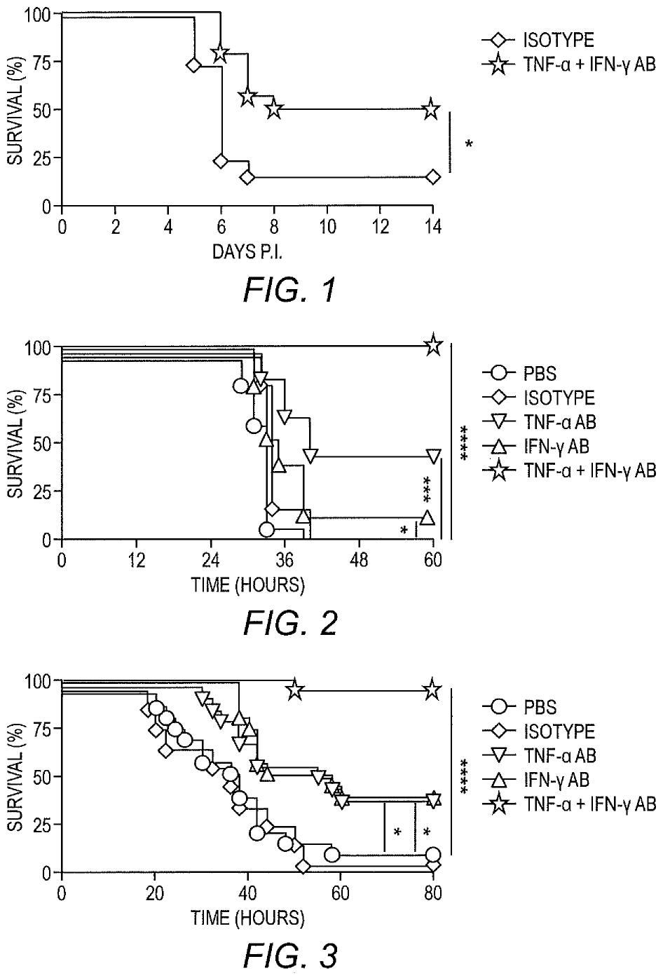

Blocking TNF-α and IFN-γ Reduces Mortality in Disease Models Associated with Cytokine Storm: SARS-CoV-2 Infection, Sepsis, and HLH. The in vitro and in vivo studies show that the synergistic activity of the pro-inflammatory cytokines TNF-α and IFN-γ mimic the symptoms of COVID-19 in mice and trigger robust cell death. These findings indicate that blocking TNF-α and IFN-γ during SARS-CoV-2 infection would prevent severe symptoms and protect against death. To understand the potential impact of blocking TNF-α and IFN-γ during disease, the efficacy of neutralizing antibodies for TNF-α and IFN-γ were tested. Mice injected with the neutralizing antibodies, but not isotype control, were protected from death during TNF-α and IFN-γ shock, indicating that these antibodies efficiently neutralized TNF-α and IFN-γ in vivo. To test the potential efficacy of this treatment during SARS-CoV-2 infection, a murine model was used. SARS-CoV-2 uses the ACE2 receptor to bind to cells and invade. Due to structural differences in mouse and human ACE2 proteins, SARS-CoV-2 is inefficient at infecting the commonly used wild-type mouse strains (McCray, et al. (2007) J. Virol. 81:813-21; Wan, et al. (2020) J. Virol. 94). Therefore, mice with the human ACE2 receptor introduced under the control of the human cytokeratin 18 (K18) promoter, also known as K18-hACE2 transgenic mice, were used. These mice are known to develop fatal disease upon SARS-CoV-2 infection (Winkler, et al. (2020) Nat. Immunol. 21:1327-1335). Mice were infected intranasally with 2×10⁴ pfu SARS-CoV-2 per mouse. By 7 days post-infection, nearly all isotype control-treated mice succumbed to the infection. Conversely, treatment with neutralizing antibodies against TNF-α and IFN-γ provided significant protection against SARS-CoV-2-induced mortality (FIG. 1).

To determine the potential therapeutic benefit of blocking this pathway in other diseases and syndromes associated with cytokine storm, the efficacy of neutralizing antibodies against TNF-α and IFN-γ were tested in other models. Poly I:C priming followed by LPS challenge recapitulates most aspects of HLH, a severe systemic inflammatory syndrome associated with life-threatening symptoms (Wang, et al. (2019) Proc. Natl. Acad. Sci. USA 116:220-2209). Treatment with the combination of TNF-α and IFN-γ blocking antibodies provided 100% protection against the lethality induced by poly I:C and LPS challenge (FIG. 2). It was subsequently investigated whether these neutralizing antibodies have therapeutic potential in a murine sepsis model. Injection of a lethal dose of LPS (20 mg/kg body weight; i.p.) induced mortality in 90% of mice by 48 hours. Neutralization of TNF-α alone or IFN-γ alone provided some protection, but combined neutralization of TNF-α and IFN-γ provided 94% protection against LPS-induced lethality (FIG. 3). These in vivo models indicate that TNF-α and IFN-γ-mediated inflammatory cell death drives pathology in not only COVID-19, but also other diseases and syndromes associated with cytokine storm, including HLH and sepsis, leading to the definition of cytokine storm as a life-threatening condition caused by excessive production of cytokines mediated by inflammatory cell death, PANoptosis.

### Example 2: Amelioration of Inflammatory Cell Death Associated with Infection or Inflammatory Disease

The results herein demonstrate that the combined expression of TNF and IFN-γ in patients who present with severe or critical symptoms associated with inflammatory disease or infections such as COVID-19 triggers inflammatory cell death, i.e., PANoptosis. Accordingly, the present invention provides for the administration of one or a combination of agents targeting the inflammatory cell death pathway as a therapeutic approach in the prevention and/or treatment of cell death and tissue damage, as well as the development of ARDS and/or multiple organ failure, in particular in conditions such as COVID-19, influenza, other viral infections, bacterial infections, sepsis, cytokine storm-associated syndromes, and inflammatory disease. Therapeutic targets of the inflammatory cell death pathway are presented in Scheme 1.

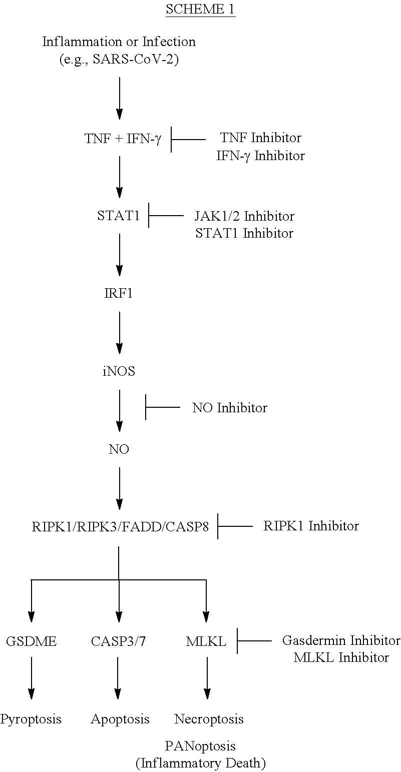

Exemplary agents that may be used alone or in combination to target one or more of the therapeutic targets of the inflammatory cell death pathway are listed in Table 1.

TABLE 1

| | |
|---|---|
| Target | Inhibitor |

Cytokine Inhibitor

| | |
|---|---|
| TNF | Infliximab, adalimumab, certolizumab, |
| | golimumab, etanercept, thalidomide, |
| | lenalidomide, pomalidomide, pentoxifylline, |
| | bupropion, delmitide |
| IFN-γ | Emapalumab, fontolizumab, AMG 811, |
| | vidofludimus, delmitide |

Signaling Inhibitor

| | |
|---|---|
| JAK1/2¹ | Ruxolitinib, Baricitinib, Tofacitinib, |
| | CYT387, AZD1480, GSK2586184, TG101348, |
| | AC-430, CEP-33779, lestaurtinib, pacritinib, |
| | BMS-911543, SB1518, R348, VX-509, GLPG0634 |
| STAT1² | Fludarabine, Nifuroxazide, pravastatin, |
| | ISS-840 |
| NO³ | L-NAME (N5-[imino(nitroamino)methyl]-L- |
| | ornithine methyl ester), 1400W (N-[[3- |
| | (aminomethyl)phenyl]methyl]-ethanimidamide), |
| | L-arginine, L-canavanine, L-NAMA, L-NNA |
| | (Nω-nitro-L-arginine), L-NIL (L-N6-(1- |
| | iminoethyl)lysine dihydrochloride), NILT (N6- |
| | iminoethyl-lysine tetrazoleamide), L-NIO |
| | (N(5)-(-iminoethyl)-L-ornithine), Vinyl-L- |
| | NIO, L-thiocitrulline, S-methyl-L- |
| | thiocitrulline, N-propyl-L-arginine, A- |
| | guanidinoglutaric acid, S-(2- |
| | aminoethyl)isothiourea, AMT (2-amino-5, 6- |
| | dihydro-6-methyl-4H-1, 3-thiazine) , |
| | Mercaptoethylguanidine, S-ethylisothiourea, |
| | S-ethyl-N-[4- |
| | (trifluoromethyl)phenyl] isothiourea, S- |
| | isopropylisothiourea, S-methylisothiourea, |
| | 1,4-BPIT (S,S′-1,4-phenylenebis (1,2- |
| | ethanediyl)bis-isothiourea), Aminoguanidine, |
| | 2-imi no-4-methylpiperdine, TRIM (1-[2- |
| | trifluoromethylphenyl] imidazole), 7- |
| | nitroindazole, 3-bromo-7-nitroindazole, BYK |
| | 191023 dihydrochloride |
| RIPK1⁴ | Nec-1s (necrostatin-1s), GSK481, GSK547, |
| | DNL104, DNL747, DNL758, R552, Compound 22, |
| | GNE684, GSK963, Compound 8, RIPA-56, 6E11, |
| | PK68, P10, necrostatin-4, necrostatin-5, |
| | necrostatin-7, necrostatin-21 |

Effector Inhibitor

| | |
|---|---|
| Gasdermin⁵ | Disulfiram, necro sulfonamide, LDC7559, |
| | Bay 11-7082 |
| CASP3 | 2-[(1-Benzyl-2-oxo-5- |
| | (thiomorpholinosulfonyl) indolin-3- |
| | ylidene]malononitrile, Z-VAD (OMe)-FMK, Z- |
| | DEVD-FMK, Caspase-3 Inhibitor III, Q-VD-OPH, |
| | Caspase 3/7 Inhibitor I, Caspase Inhibitor X, |
| | Ivachtin, DICA, NSCI, Ac-ATS010-KE, AC- |
| | DEVD-CHO |
| CASP7 | DICA, Caspase 3/7 Inhibitor I, Caspase |
| | Inhibitor X, Z-DEVD-FMK |
| MLKL | Necrosulfonamide, URMC-099, AZ7550, |
| | LY-364947 |

¹Furumoto & Gadina (2013) BioDrugs 27(5): 431-438.
²Miklossy, et al. (2013) Nat. Rev. Drug Discov. 12 (8): 611-629.
³Janakiram & Rao (2012) Future Med. Chem. 4 (17): 2193-2204.
⁴Mifflin, et al. (2020) Nat. Rev. Drug Discov. 9:553-571.
⁵Xia, et al. (2020) Cold Spring Harb. Perspect. Biol. 12(3): a036400.

In general, therapeutic combinations can include, but are not limited to: (a) two or more cytokine inhibitors, (b) two or more signaling inhibitors, (c) two or more effector inhibitors, (d) at least one cytokine inhibitor and at least one signaling inhibitor, (e) at least one cytokine inhibitor and at least one effector inhibitor, (f) at least one signaling inhibitor and at least one effector inhibitor, or (g) at least one cytokine inhibitor, at least one signaling inhibitor and at least one effector inhibitor. More specifically, therapeutic combinations can include, but are not limited to: (a) a TNF inhibitor and an IFN-γ inhibitor; (b) a TNF inhibitor and an inhibitor of at least one of JAK1/2, STAT1, NO or RIPK1; (c) two or more inhibitors, each of which inhibits at least one of JAK1/2, STAT1, NO or RIPK1; (d) an IFN-γ inhibitor and an inhibitor of at least one of JAK1/2, STAT1, NO or RIPK1; (e) a TNF inhibitor and an inhibitor of at least one of Gasdermin, CASP3, CASP7 or MLKL; (f) an IFN-γ inhibitor and an inhibitor of at least one of Gasdermin, CASP3, CASP7 or MLKL; (g) an inhibitor of at least one of JAK1/2, STAT1, NO or RIPK1 and an inhibitor of at least one of Gasdermin, CASP3, CASP7 or MLKL; or (h) two or more inhibitors, each of which inhibits at least one of Gasdermin, CASP3, CASP7 or MLKL.

### Example 3: Agonism of TNF and IFN-γ Promotes Inflammatory Cell Death

Mice. The NOD.Cg-Prkdc^{scid} Il2rg^{tm1Wjl}/SzJ (NSG, Jackson Laboratory) mice were acquired and maintained at St. Jude Children's Research Hospital (SJCRH). All mice were housed at SJCRH animal facilities under strict specific pathogen-free conditions (sterilized water, food, bedding and cages), and aseptic handling techniques were used to avoid any unwanted infections. Animal studies were conducted under protocols approved by SJCRH's committee on the use and care of animals.

Analysis of TCGA Cytokine Expression Data from Patients with Colon Cancer. Cytokine expression and survival data for patients with colorectal cancer from the Cancer Genome Atlas Program were downloaded from Human Protein Atlas (Uhlen, et al. (2017) Science 357:eaan2507). Normal expression data were downloaded from Genomic Data Commons Data Portal of National Cancer Institute (Grossman, et al. (2016) N. Engl. J. Med. 375:1109-1112). The heatmap was generated using Morpheus. Survival analysis and graph generation was done in GraphPad Prism.

Cell Culture and Stimulation Methods. The fully authenticated human NCI-60 cancer cell lines (NCI, Bethesda, MD) used in this study were cultured in RPMI media (Corning) supplemented with 10% FBS and 1% penicillin and streptomycin. Cancer cells were seeded a day before stimulation. On the day of stimulation, the cells were washed once with warm and sterile DPBS, followed by treatment with different cytokines for 48 hours. The concentration of the cytokines used were 25 ng/mL of IL-6 (Peprotech), IL-8 (Peprotech), IL-18 (R&D), IL-15 (Peprotech), IL-1α (Peprotech), IL-1β (Peprotech), IL-2 (Peprotech), TNF-α (Peprotech), and IFN-γ (Peprotech). For the inhibition of nitric oxide (NO), cancer cells were co-treated with 1 mM of L-NAME hydrochloride (TOCRIS) or 100 μM of 1400W dihydrochloride (Enzo Life Sciences) along with the cytokines. For inhibition of the JAK signaling pathway, the cancer cells were co-treated with 5 μM of baricitinib (Achemblock) all through the experiment.

Generation of IRF1-Knockout HCT116 Cells Using CRISPR-Cas9 System. HCT116 colon cancer cells (ATCC® CCL-247™; fully authenticated by ATCC) were transduced with lentiviral Streptococcus pyogenes Cas9-GFP and flow sorted for GFP-positive cells with optimal expression levels of Cas9-GFP. Lentiviral particles expressing two different IRF1 guide RNAs (gRNAs, 5′-CTTGGCAGCATGCTTCCATGGG-3′ (SEQ ID NO:1) and 5′-TTGCTCTTAGCATCTCGGCTGG-3′ (SEQ ID NO:2)) were generated by using the HEK293T packaging system. The HEK293T cells were transfected with IRF1 guide RNAs, along with the packaging plasmids pPAX2 and pMD2. Lentiviral particles were collected 48 hours post-transfection. The HCT116 cells that express the Cas9-GFP protein were transduced with IRF1 gRNA lentiviral particles in combination with 8 μg/ml polybrene for 24 hours. The HCT116 cells carrying successful integration of the gRNAs were selected using puromycin (2.5 μg/ml). The IRF1 knockout and the corresponding control Cas9-expressing wild-type HCT116 cells were used in further experiments.

Cell Death Analysis. The kinetics of cell death were measured using a two-color INCUCYTE® S3 imaging system (Essen Biosciences). Different lines of NCI-60 cancer cells were seeded in 12-well (0.25×10⁶ cells/well) or 24-well (0.125×10⁶ cells/well) cell culture plates and stimulated with indicated cytokines in the presence of the cell-impermeable DNA binding fluorescent dye SYTOX® Green (Life Technologies, 20 nM) following the manufacturer's protocol. A series of images were acquired at 1-hour time intervals for up to 48 hours post-treatment with a 20× objective and analyzed using the INCUCYTE® S3 software, which allows precise analysis of the number of dye-positive dead cells present in each image. A minimum of three images per well was taken for the analysis of each time point. Dye-positive dead-cell events for each of the cancer cells were plotted using GraphPad Prism version 5.0 software.

Western Blot Analysis. For the western blot analyses of caspases, cancer cells were seeded a day before stimulation at a density of 0.5×10⁶ cells/well in 6-well cell culture plates. The proteins from the indicated cell types were collected by combining cell lysates with culture supernatants with caspase lysis buffer (with 1× protease inhibitors, 1× phosphatase inhibitors, 10% NP-40, and 25 mM DTT) and 4× sample loading buffer (containing SDS and 2-mercaptoethanol). For the western blot analysis of all other signaling proteins, the cells were lysed in RIPA buffer, supplemented with protease inhibitor and phosphoStop as per the manufacturer's instructions and sample loading buffer. Samples were denatured by boiling for 10 minutes at 100° C. and separated using SDS-PAGE followed by the transfer on to Amersham HYBOND® P polyvinylidene difluoride membranes (GE Healthcare Life Sciences) and immunoblotted with primary antibodies against IRF1 (rabbit monoclonal antibody (mAb); Cell Signaling Technology), STAT1 (rabbit mAb; Cell Signaling Technology), caspase-1 (rabbit polyclonal antibody (pAb); Proteintech), caspase-3 (rabbit pAb; Cell Signaling Technology), cleaved caspase-3 (rabbit pAb; Cell Signaling Technology), caspase-7 (rabbit pAb; Cell Signaling Technology), gasdermin D (rabbit pAb; Cell Signaling Technology), GSDME/DFNA5 (rabbit mAb; Abcam), caspase-8 (mouse mAb, clone 12F5; Enzo Life Sciences), MLKL (rabbit mAb, clone EPR17514; Abcam), RIPK3/RIP3 (rabbit pAb; Novus Biologicals) and β-actin (rabbit mAb, 13E5; Cell Signaling Technology) followed by secondary anti-rabbit or anti-mouse HRP antibodies (Jackson ImmunoResearch Laboratories), as previously described (Tweedell, et al. (2020) Nature Protocols 15:3284-3333).

Membrane Proteins and MLKL Oligomerization Methods. The membrane proteins were isolated using the Thermo Scientific Mem-PER Plus Membrane Protein Extraction Kit. For western blot analysis of MLKL oligomerization (activation), the cells were lysed in NP-40 lysis buffer (1.0% NP-40, 150 mM NaCl, and 50 mM HEPES) containing complete protease inhibitors and phosphoSTOP (Roche) and divided in two fractions. One half of the lysate was mixed with a sample loading dye containing the disulfide bond-reducing reagent 2-BME (2-Mercaptoethanol, 25 mM) and the other half with a sample buffer without any reducing agents. Both of these lysate preparations were boiled for minutes at 100° C. and subjected to immunoblotting analysis of total MLKL protein.

NSG Mouse Xenograft Tumor Model. The NSG mice (NOD/SCID-gamma null mouse strain that lacks IL-2-γ chain) were used as the model recipients for the subcutaneous transplantation of human COLO-205 cancer cells. Briefly, 8- to 10-week-old NSG mice were shaved on their lower backs and transplanted with 2×10⁶ COLO-205 cells in 200 μl of medium prepared by mixing (1:1) RPMI and a solubilized basement membrane preparation sold under the tradename MATRIGEL® (BD Bioscience). The mice were monitored regularly for engraftment and growth of COLO-205 cancer cell tumors. The recipient mice were divided into four groups and treated intratumorally with injection of the indicated cytokines or PBS (groups: (i) PBS, (ii) TNF-α, (iii) IFN-γ, or (iv) TNF-α(and IFN-γ) on day 12, 14 and 16 post-transplantation of the tumor cells. The tumor growth was monitored and measured using digital calipers, and the volume of the tumors were calculated using the formula: volume=(length×width²)×½.

Statistical Analysis. GraphPad Prism version 5.0 and 8.0 software were used for data analyses. Data are shown as mean±SEM. Statistical significance was determined by t tests (two-tailed) or Log-rank (Mantel-Cox) tests for two groups, and one-way ANOVA (with Dunnett's or Tukey's multiple comparisons tests) for three or more groups. The number of experimental repeats and technical replicates are indicated in the corresponding figure legends; n in the figure legends represents the number of biological replicates used in the experiments.

TNF-α and IFN-γ Cooperatively Induce Cancer Cell Death. Expression and modulation of cytokines have critical functions associated with tumor growth and therapeutic outcomes (Karki & Kanneganti (2019) Nat. Rev. Cancer 19:197-214; Grivennikov, et al. (2010) Cell 140:883-899; Landskron, et al. (2014) J. Immunol. Res. 2014:149185). To determine which proinflammatory cytokines are highly modulated in tumors, a publicly available TCGA dataset from healthy volunteers and patients with different stages of colorectal cancer (CRC; Stage I to IV, low to high grade tumors) was re-analyzed. The analyses showed altered expression of TNF-α, IFN-γ, IL-1α, IL-1β, IL-18, IL-6, IL-8 and IL-15 in the tumor microenvironment (Table 2).

TABLE 2

| | | Stage | | | |
|---|---|---|---|---|---|
| Gene | Normal | I | II | III | IV |
| TNF-α | +++ | + | −− | −−− | − |
| IFN-γ | ++ | +++ | +++ | +/− | −−− |
| IL-1α | −− | −−− | +++ | −− | − |
| IL-1β | +++ | −−− | ++ | − | −− |
| IL-18 | +++ | − | − | −−− | −−− |
| IL-6 | −− | −−− | +++ | −− | + |
| IL-8 | +++ | −−− | ++ | −− | −− |
| IL-15 | +++ | + | − | − | −−− |

Relative abundance,
maximum = +++,
minimum = −−−

It was also observed that the decreased expression of proinflammatory cytokines was often associated with a decreased probability of survival. These findings indicated a strong inverse correlation between proinflammatory cytokine profiles and cancer progression. However, the role of specific cytokines in modulating tumorigenesis was not clear. It is possible that impaired cell death in the absence of proinflammatory cytokines contributes to the reduced probability of survival. To investigate this possibility, the human colon cancer cell line HCT-116 was treated with the proinflammatory cytokines that were differentially modulated in the tumor microenvironment of patients with CRC. Cell death was not detected in response to any of these cytokines individually. However, combining all the selected cytokines, induced robust cancer cell death, indicating that synergistic cytokine signaling is required for this process. In addition, it was found that TNF-α and IFN-γ alone or the cytokine combination that contained TNF-α and IFN-γ, but not a cytokine combination which lacks both TNF-α and IFN-γ, induced human cancer cell death. These results indicate that TNF-α and IFN-γ are required to trigger cancer cell death in HCT-116.

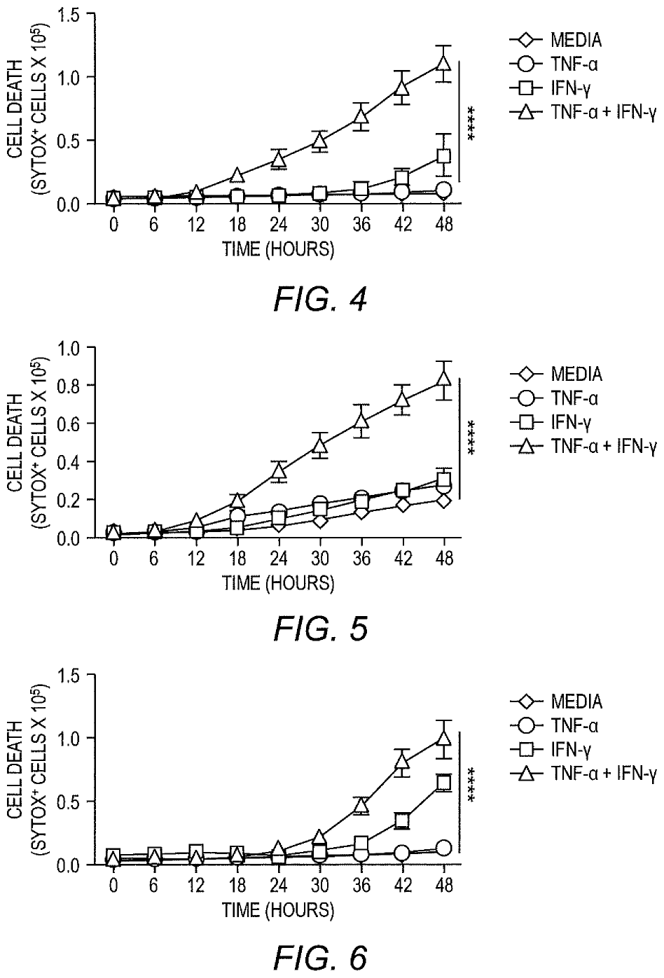

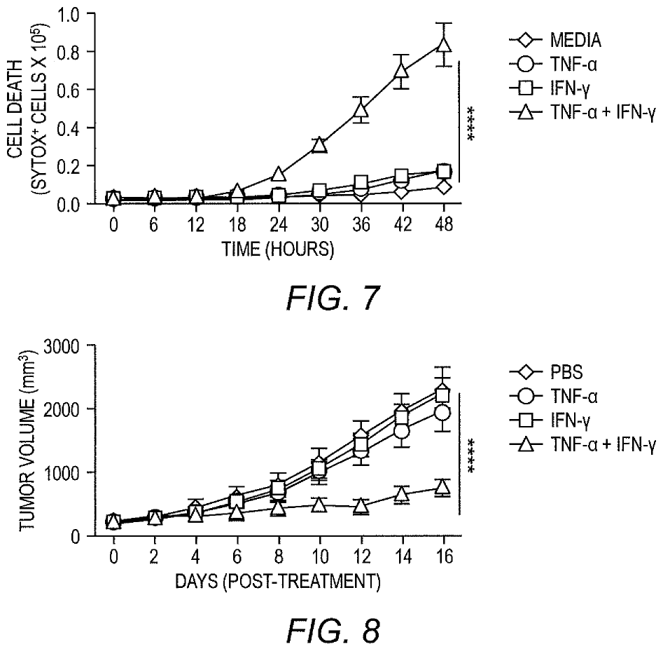

The efficacy of the TNF-α and IFN-γ combination in triggering cell death was subsequently assessed in a broad range of human colon cancer cell lines. The NCI-60 colon cancer cell lines, HT-29 (FIG. 4), SW-620 (FIG. 5), HCC2998 (FIG. 6), and COLO-205 (FIG. 7), were susceptible to TNF-α and IFN-γ co-treatment-mediated cell death. To study the relative contribution of TNF-α or IFN-γ in inducing cell death, the NCI-60 colon cancer lines were treated with TNF-α or IFN-γ alone, or in combination. The cell death induced by co-treatment of TNF-α and IFN-γ was more robust than that induced by individual treatment with TNF-α or IFN-γ (FIG. 4, FIG. 5, FIG. 6 and FIG. 7). These findings were extended to other cancer lines from the NCI-60 panel. Comprehensive screening demonstrated that TNF-α and IFN-γ co-treatment induced cell death in a range of cancer cells, including the NCI-60 melanoma lines SK-MEL-2, M14, SK-MEL-5 and UACC-62; lung cancer lines HOP-92 and H226; and leukemia lines HL-60 and RPMI-8226. These findings indicate that various cancer cells may have varying sensitivities to TNF-α and IFN-γ individually, but the combined treatment triggers robust cell death across a wide range of cancer cells.

TNF-α and IFN-γ Induce PANoptosis in Cancer Cells. As demonstrated herein, synergism of TNF-α and IFN-γ induces inflammatory cell death, PANoptosis, by activating gasdermin E (GSDME), caspase-8, -3 and -7, and MLKL in murine bone marrow-derived macrophages (BMDMs). To determine whether PANoptosis was activated in cancer cells, the biochemical markers of PANoptosis were analyzed in colon cancer lines. Consistent with the cell death data, it was found that the combination of TNF-α plus IFN-γ activated PANoptosis in the HCT-116 colon cancer cells, while either cytokine treatment alone induced only weak activation of these biochemical markers.

The effect of the TNF-α and IFN-γ combination was subsequently evaluated in different cancer cell lines to characterize the mechanism of cell death. Without cytokine treatment, robust expression of caspase-1 was detected in the colon cancer lines HT-29 and COLO-205, but not in SW-620 or HCC2998. TNF-α and IFN-γ treatment further upregulated the expression of caspase-1 in all the colon cancer lines tested. Active caspase-1 was also detected in TNF-α and IFN-γ-treated COLO-205 cells, indicating that pyroptosis may be occurring in these cells. However, the expression of the pyroptotic executioner GSDMD was not observed in COLO-205 cells, suggesting that COLO-205 cells cannot undergo GSDMD-mediated pyroptosis. Similar to the varied caspase-1 expression, the expression of GSDMD and GSDME was also varied across the different cell lines. Furthermore, robust activation of GSDME, another executioner of pyroptosis known to be cleaved by caspase-3 (Wang, et al. (2017) Nature 547:99-103; Rogers, et al. (2017) Nat. Commun. 8:14128), was observed in response to TNF-α and IFN-γ treatment in HCT-116 and SW-620 cells, whereas HCC2998 and COLO-205 cancer cells showed only minor cleavage, confirming that pyroptotic effectors are activated in these cells. In addition, proteolytic cleavage of apoptotic caspases including the upstream caspase-8 and the downstream caspase-3 and -7 was observed in all 5 colon cancer lines, indicating a universal susceptibility of these cancer cell lines to TNF-α and IFN-γ treatment-induced apoptotic effector activation. Next, the role of TNF-α and IFN-γ in inducing necroptotic effectors was examined by assessing the oligomerization of MLKL as a measure of its activation. Higher order oligomers of MLKL were observed in cells treated with TNF-α and IFN-γ. Additionally, the expression of RIPK3 and MLKL was intact in all these colon cancer lines. Furthermore, TNF-α and IFN-γ co-treatment induced PANoptosis in a range of other cancer cells, including the NCI-60 melanoma lines SK-MEL-2, M14, SK-MEL-5 and UACC-62; lung cancer lines HOP-92 and H226, and leukemia lines HL-60 and RPMI-8226. Overall, the contemporaneous activation of pyroptotic, apoptotic, and necroptotic effectors observed in these cell lines indicate that TNF-α and IFN-γ induce robust PANoptosis in a wide variety of cancer cells.

TNF-α and IFN-γ Drive IRF1-Dependent, but iNOS-Independent Cancer Cell Death. As described herein TNF-α plus IFN-γ treatment induces death of immune cells in murine BMDMs through the JAK/STAT1-IRF1-iNOS signaling axis. To investigate whether TNF-α and IFN-γ treatment utilizes a similar mechanism to induce human cancer cell death, HCT-116 cells deficient in IRF1 were generated, and HCT-116 control and IRF1-deficient HCT-116 colon cancer cells were treated with the combination of TNF-α and IFN-γ. The cell death induced by TNF-α and IFN-γ was significantly reduced in IRF1-deficient HCT-116 colon cancer cells compared to HCT-116 control cancer cells. Importantly, the reduced cleavage of GSDMD and GSDME and caspase-8, -3 and -7, and the reduction in high order oligomers of MLKL from the cell membrane fraction in IRF1-deficient HCT-116 colon cancer cells confirms that IRF1 is required for TNF-α plus IFN-γ treatment-induced PANoptosis.

Signaling through JAK-STAT1-IRF1 has been shown herein to be required for NO production, which in turn induces PANoptosis in macrophages. To investigate the role of the JAK-STAT1 pathway in PANoptosis induced by TNF-α and IFN-γ, cancer cells were treated with the JAK inhibitor baricitinib. Baricitinib-treated cancer cells showed reduced cell death, indicating that the JAK-STAT1 pathway is required for cancer cell death. To further investigate the role of NO in this process, cancer cells were treated with the NO inhibitors L-NAME (NOS inhibitor) or 1400W (iNOS inhibitor) and cell death was monitored. In contrast to their effect in macrophages, NO inhibitors failed to inhibit the cancer cell death induced by TNF-α plus IFN-γ treatment both in colon cancer and melanoma cell lines. Together, these results indicate that the JAK-STAT1-IRF1 signaling axis plays a key role in driving TNF-α and IFN-γ treatment-induced cancer cell PANoptosis, while NO is dispensable for this process.

TNF-α Plus IFN-γ Suppresses Tumor Growth in vivo. The therapeutic potential of TNF-α and IFN-γ to prevent tumor development was evaluated in tumor-bearing NSG mice (FIG. 8). It was observed that intra-tumoral administration of TNF-α alone provided a minor but significant reduction in tumor weight, while combined intra-tumoral injection of TNF-α and IFN-γ resulted in a larger reduction in tumor weight and significantly reduced the tumor volume (FIG. 8). Together, these findings demonstrate that administration of IFN-γ promotes robust JAK-STAT-dependent IRF1 expression and potentiates TNF-α-mediated cell death, PANoptosis, and that the TNF-α and IFN-γ combination was superior to single agents alone in treating the established tumors. These findings demonstrate the applicability of TNF-α and IFN-γ-mediated cell death for the treatment of tumors in vivo.

What is claimed is:

1. A method for treating or mitigating COVID-19 comprising administering to a subject in need of treatment an effective amount of a Tumor Necrosis Factor (TNF) inhibitor and an Interferon gamma (IFN-γ) inhibitor thereby treating or mitigating COVID-19.

2. The method of claim 1, wherein the TNF inhibitor is infliximab, adalimumab, certolizumab, golimumab, etanercept, thalidomide, lenalidomide, pomalidomide, pentoxifylline, bupropion, or delmitide.

3. The method of claim 1, wherein the IFN-γ inhibitor is emapalumab, fontolizumab, AMG 811, vidofludimus, or delmitide.

4. A kit for treating or mitigating COVID-19 comprising a Tumor Necrosis Factor (TNF) inhibitor and an Interferon gamma (IFN-γ) inhibitor.

5. The kit of claim 4, wherein the TNF inhibitor is infliximab, adalimumab, certolizumab, golimumab, etanercept, thalidomide, lenalidomide, pomalidomide, pentoxifylline, bupropion, or delmitide.

6. The kit of claim 4, wherein the IFN-γ inhibitor is emapalumab, fontolizumab, AMG 811, vidofludimus, or delmitide.

7. A method for treating or preventing inflammatory cell death associated with an infection or inflammatory condition comprising administering to a subject with an infection or inflammatory condition an effective amount of a TNF inhibitor, an IFN-γ inhibitor or a combination thereof thereby treating or preventing inflammatory cell death associated with the infection or inflammatory condition.

8. The method of claim 7, wherein the TNF inhibitor is infliximab, adalimumab, certolizumab, golimumab, etanercept, thalidomide, lenalidomide, pomalidomide, pentoxifylline, bupropion, or delmitide.

9. The method of claim 7, wherein the IFN-γ inhibitor is emapalumab, fontolizumab, AMG 811, vidofludimus, or delmitide.

10. The method of claim 7, wherein the infection is a bacterial infection, a fungal infection, a parasitic infection, or a viral infection.

11. The method of claim 10, wherein the viral infection is an influenza infection or a coronavirus infection.

12. The method of claim 11, wherein the coronavirus infection is SARS-CoV-2.

13. The method of claim 7, wherein the inflammatory condition is sepsis, hemophagocytic lymphohistiocytosis or TNF- and IFN-γ-associated cytokine shock and inflammation.

14. A kit for treating or preventing inflammatory cell death associated with an infection or inflammatory condition comprising a Tumor Necrosis Factor (TNF) inhibitor and an Interferon gamma (IFN-γ) inhibitor.

15. The kit of claim 14, wherein the TNF inhibitor is infliximab, adalimumab, certolizumab, golimumab, etanercept, thalidomide, lenalidomide, pomalidomide, pentoxifylline, bupropion, or delmitide.

16. The kit of claim 14, wherein the IFN-γ inhibitor is emapalumab, fontolizumab, AMG 811, vidofludimus, or delmitide.


SEQUENCE LISTING (2 sequences)

SEQ ID NO: 1 - DNA, 22 bp, Artificial Sequence
  Synthetic oligonucleotide
cttggcagcatgcttccatggg

SEQ ID NO: 2 - DNA, 22 bp, Artificial Sequence
  Synthetic oligonucleotide
ttgctcttagcatctcggctgg